(19) United States
(12) Patent Application Publication
(10) Pub. No.: US 2014/0314676 A1
(43) Pub. Date: Oct. 23, 2014

(54) METHODS OF TREATMENT WITH DEFERIPRONE

(71) Applicants: APOTEX TECHNOLOGIES INC., Toronto (CA); SUNNYBROOK RESEARCH INSTITUTE, Toronto (CA); CHILDREN'S HOSPITAL LOS ANGELES, Los Angeles, CA (US)

(72) Inventors: Michael Spino, Pickering (CA); John Chalmers Connelly, Kettleby (CA); John Charles Wood, Pasadena, CA (US); Graham Arnold Wright, Toronto (CA); Nilesh Ramesh Ghugre, Toronto (CA)

(21) Appl. No.: 14/358,953

(22) PCT Filed: Nov. 16, 2012
(86) PCT No.: PCT/US2012/065663

§ 371 Date: May 16, 2014

Related U.S. Application Data
(60) Provisional application No. 61/561,692, filed on Nov. 18, 2011.
(60) Provisional application No. 61/591,854, filed on Jan. 27, 2012.

(51) Int. Cl. A61K 31/4412; A61K 45/06

(52) U.S. Cl. CPC A61K 31/4412; A61K 45/06
USPC 424/9.3; 514/348; 424/94.64; 424/94.63; 514/383; 514/301; 514/312; 424/133.1; 424/9.1

(57) ABSTRACT

The current application is directed to methods of treating or ameliorating myocardial ischemia, an acute coronary event, and a myocardial reperfusion injury comprising administering a therapeutically effective amount of deferiprone or a pharmaceutically acceptable salt thereof to a patient in need thereof. The application is also directed to reducing the risk for myocardial reperfusion injury as well as promoting the beneficial remodeling of cardiac tissue in a patient, comprising administering a therapeutically effective amount of deferiprone or a pharmaceutically acceptable salt thereof to a patient before, during or after reperfusion therapy. The application also includes methods of selecting a patient for treatment of reperfusion injury and subsequently treating the selected patient.

### BACKGROUND OF THE INVENTION

1. Field of the Invention

The invention provides methods of reducing the risk of a myocardial injury, e.g., intramyocardial hemorrhage, cardiac edema, reperfusion arrhythmias, adverse remodeling, and/or ischemic damage, following a myocardial infarction; treating, preventing or ameliorating myocardial ischemia, an acute coronary event, or reperfusion injury; and promoting the revascularization and beneficial remodeling of cardiac tissue, comprising administering a therapeutically effective amount of deferiprone or a pharmaceutically acceptable salt thereof to a patient in need thereof. The invention also provides methods for selecting a patient for treatment with deferiprone or a pharmaceutically acceptable salt thereof.

2. Background

Heart disease is one of the leading causes of death in the United States and Canada responsible for nearly 25% and 22% of all deaths, respectively, based on a 2007 report by Statistics Canada (1). Reportedly, 1.5 million people suffer from heart attacks annually in the United States of which about 500,000 events lead to death. In 2006, 631,636 people died of heart disease, making heart disease the leading cause of death for both men and women. Coronary heart disease is the most common type of heart disease. Every year about 785,000 Americans have a first heart attack, and another 470,000 who have already had one or more heart attacks have another attack. It was estimated that in 2010, heart disease cost the United States about $316.4 billion in health care services, medications, and lost productivity.

Acute myocardial infarction occurs due to cessation of blood flow into the heart muscle, thereby resulting in irreversible necrosis in the region supplied by the concerned coronary artery (5). Reperfusion therapy is standard in modern treatment of acute myocardial infarction. For example, patients with suspected acute myocardial infarction and/or ST segment elevation (STEMI) are presumed to have an occlusive thrombosis in a coronary artery, and they are therefore candidates for immediate reperfusion, either with thrombolytic therapy or percutaneous coronary intervention (PCI), and when these therapies are unsuccessful the next intervention is usually bypass surgery.

While reperfusion is favorable in terms of myocardial salvage, it may result in additional cardiac damage rivaling that of the initial event, i.e., ‘reperfusion injury’ (RI) (10). RI has been associated with worsening or expansion of the prior ischemic damage resulting in microvascular dysfunction arising from endothelial cell damage, stunning, reperfusion arrhythmias, and further myocyte death; a contributor to these effects is free radical generation. Intracellular and interstitial edema is also associated with RI in acute myocardial infarction (AMI) arising from a local inflammatory reaction (11).

In addition, a phenomenon called ‘no-reflow’ is often encountered, which is typically caused by ischemia-induced microvascular obstruction (MVO) and injury and has been correlated with adverse left ventricular (LV) remodeling and poor patient outcome (12). Furthermore, reperfusion coupled with a severe initial ischemic insult may also result in intramyocardial hemorrhage (13), which in association with MVO is believed to be an independent predictor of adverse remodeling (14).

Following reperfusion, patients are typically treated with anti-platelets, statins, angiotensin-converting enzyme (ACE) inhibitors and beta-blockers that have shown promise in limiting RI, infarct size, and adverse LV remodeling (15, 16). However, even with current pharmacotherapies morbidity and mortality remain high. A significant number of post-AMI patients still go on to develop LV enlargement and heart failure, particularly the ones who develop large transmural infarcts with microvascular dysfunction (17, 18); heart transplantation or ventricular assist devices may be required in some cases. Furthermore, since remodeling is a complex process, treating one reparative pathway may have deleterious consequences on another (19). Thus, the effects of novel protective pathways involving immune response, the reperfusion injury salvage kinase (RISK) pathway, mitochondrial permeability transition pore (PTP), etc. are still under investigation (10, 20). Although several investigators have reported the success of ‘ischemic pre- and post-conditioning’ in patients after AMI (21, 22), the findings of a recent study have been negative (23). Thus, translation of cardioprotection into clinical practice has generally been unsuccessful.

The presentation of intramyocardial hemorrhage as a consequence of reperfusion injury in AMI has been well documented in both humans (14, 33) and animal models (25, 26, 34, 35). Oxidative stress, calcium overload, pH fluctuation, increased inflammation, and mitochondrial damage are the predominant components of reperfusion injury that result in cellular and vascular damage. Patients with hemorrhagic infarcts appear to be at high risk, with poor long-term outcomes (14, 47). The changing appearance of tissue hemorrhage has been extensively studied in the case of brain hemorrhage (39). Degradation products of hemoglobin have been associated with increased brain edema, neuronal damage and neurological defects (40, 41). The evolution of hemorrhagic infarcts has not been well investigated in AMI and the associated effects on inflammation and oxidative stress are currently unclear.

A key component of a biological system's immune response to tissue injury is inflammation, which is triggered to aid clearing of the necrotic debris, allowing the process of tissue healing to begin (48-51). In the acute phase of infarction, the humoral inflammatory stress response induces an upregulation of pro-inflammatory cytokines such as TNF-α, IL-1 and IL-6 in both infarcted (50-fold) as well as remote (15-fold) myocardium; the levels typically return to baseline after 1 week. However, if the infarction is large, cytokine expression and upregulation may be persistent, leading to a cascade activation that extends further into the peri-infarct and remote territories, and to unfavorable remodeling and worse clinical outcomes (52). TNF-α has been implicated in mediating inflammatory injury by suppressing cardiac contractility, enhancing apoptosis, and interfering with collagen synthesis (53-56). IL-10 is a potent anti-inflammatory cytokine that is expressed by lymphocytes and monocytes (57, 58); it can inhibit the production of TNF-α, IL-1 and IL-6 and is speculated to help in stabilization of the extracellular matrix (59). In addition, toll-like receptor (TLR) mediated pathways, the complement cascade, reactive oxygen species (ROS), and the chemokine family are activated and play an important role in the inflammatory cascade and healing process (50). At the same time, sequential infiltration of blood-derived cells like platelets, neutrophils, mononuclear cells, mast cells, fibroblasts and vascular cells is an integral part of the reparative process following infarction.

Although several anti-inflammatory strategies have demonstrated a reduction in infarct size and attenuation of adverse remodeling in experimental models of AMI (60), their translation into clinical practice has been controversial (50, 51) as they may actually promote LV remodeling and arrhythmias (61, 62). The multifunctional and redundant nature of cytokines has resulted in unpredictable effects when probing cytokine-mediated therapeutic strategies (50).

The use of iron chelation therapy with deferoxamine (DFO) has previously been explored as a means to decrease free-radical damage during cardiac ischemia/reperfusion. Cardioprotection has been demonstrated in isolated rat and rabbit heart preparations as well as several large animal studies. Deferoxamine pretreatment prior to cardiopulmonary bypass has been evaluated in three small clinical trials; deferoxamine lowered free-radical mediated lipid damage and white blood cell activation, and improved myocardial performance. (92, 93, 94). However, in myocardial infarction, no clinical benefit has been demonstrated with DFO. See, e.g., Chan et al., Circ. Cardiovasc Interv. 5:270-278 (2012). In Chan et al., DFO was effective in reducing reactive oxygen species (ROS) (F2-isoprostane), but had no significant effect on infarct size, creatinine kinase or Troponin-1.

While deferoxamine avidly binds circulating iron species, its large size and hydrophilicity limit cardiac myocyte penetration, potentially accounting for the variable efficacy reported in previous investigations (95, 96, 97, 98). The long delay between infusion initiation and peak drug levels in the myocardium may prevent its utility in acute coronary syndromes where presentation is unplanned. Lastly, it can only be administered parenterally, making it ill-suited for outpatient use.

A study published more than two decades ago using a Langendorff heart preparation (ex vivo) suggested that deferiprone might be helpful in post-ischemic cardiac protection (van der Kraaij A M, et al., Circulation 80:158-64 (1989)). While the study demonstrated a reduction in free radical activity in the Langendorff model, at a constant deferiprone infusion of 50 μM, the intravenous dose required to achieve such a constant infusion rate in a human would be expected to be toxic to non-iron loaded humans. Furthermore, the study did not report on any changes in ischemic damage, intramyocardial hemorrhage or cardiac edema. Thus, if there is to be net benefit to a patient, there is a need to introduce a method of treatment that exceeds theoretical benefits of reduced oxidative stress secondary to reperfusion, such as the theoretical benefits advocated for decades with vitamins, antioxidants and nutraceuticals, which have never demonstrated any actual therapeutic relevance for such a use.

Thus, there is a need to develop methods of treating and/or reducing the risk of intramyocardial hemorrhage, cardiac edema, reperfusion arrhythmias, adverse cardiac remodeling, and ischemic damage in a subject, e.g., a human, who has suffered an acute coronary event, i.e., acute myocardial infarction.

Citation or discussion of a reference herein shall not be construed as an admission that such is prior art to the present invention.

### BRIEF SUMMARY OF THE INVENTION

As discussed herein, the iron chelator, deferiprone, is uniquely suited to reduce iron-mediated damage following ischemia reperfusion. Deferiprone has superior myocyte penetration because it has a very small molecular weight (139 Daltons), is sufficiently hydrophilic to be orally absorbed and sufficiently lipophylic to permeate membranes, and maintain a neutral charge in its bound and unbound state, distinguishing it from the other currently marketed iron chelators. Deferiprone can be administered intravenously, yielding rapid cardiac protection, or be given orally for convenient chronic administration.

One aspect of the invention is directed to a method for treating or ameliorating myocardial ischemia or an acute coronary event, comprising administering a therapeutically effective amount of deferiprone or a pharmaceutically acceptable salt thereof to a patient in need thereof.

Another aspect of the invention is directed to a method for treating or ameliorating an intramyocardial hemorrhage or the damage from an intramyocardial hemorrhage, comprising administering a therapeutically effective amount of deferiprone or a pharmaceutically acceptable salt thereof to a patient in need thereof, wherein the patient is being treated for myocardial ischemia or an acute coronary event.

Another aspect of the invention is directed to a method for treating or ameliorating edema, comprising administering a therapeutically effective amount of deferiprone or a pharmaceutically acceptable salt thereof to a patient in need thereof, wherein the patient is being treated for myocardial ischemia or an acute coronary event.

In one embodiment, the myocardial ischemia or acute coronary event is an acute myocardial infarction or a ST-segment elevation myocardial infarction (STEMI).

In further embodiments, the patient is given a reperfusion therapy, e.g., a percutaneous coronary intervention (PCI) or a thrombolytic therapy. In one embodiment, the patient who is given reperfusion therapy is being treated with a method disclosed herein, e.g., for an intramyocardial hemorrhage or the damage from an intramyocardial hemorrhage, or for an edema. In further embodiments, the patient is treated with a method disclosed herein before, during or after the patient is given reperfusion therapy.

In certain embodiments, the deferiprone or pharmaceutically acceptable salt thereof is administered at a time before, during or after the patient is given the reperfusion therapy. In another embodiment, the deferiprone or pharmaceutically acceptable salt thereof is administered after the patient is given the reperfusion therapy.

Another aspect of the invention is directed to a method for treating or ameliorating a myocardial injury, comprising administering a therapeutically effective amount of deferiprone or a pharmaceutically acceptable salt thereof to a patient during or after a reperfusion therapy.

In one embodiment, the myocardial injury is selected from the group consisting of intramyocardial hemorrhage, cardiac edema, reperfusion arrhythmias, ischemic damage, and any combination thereof.

In another embodiment, the reperfusion therapy is a percutaneous coronary intervention (PCI), e.g., coronary angioplasty or insertion of a stent, or a thrombolytic therapy, e.g., administering a thrombolytic agent selected from the group consisting of streptokinase, urokinase, alteplase, recombinant tissue plasminogen activator (rtPA), reteplase, tenecteplase, and any combination thereof.

In certain embodiments, the patient further has an ischemia-induced microvascular obstruction.

Another aspect of the invention is directed to a method of reducing the risk for a myocardial injury, comprising administering a therapeutically effective amount of deferiprone or a pharmaceutically acceptable salt thereof to a patient who is at risk of myocardial injury.

Another aspect of the invention is directed to a method for reducing the risk for intramyocardial hemorrhage or damage resulting therefrom, cardiac edema, or reperfusion arrhythmias, comprising administering a therapeutically effective amount of deferiprone or a pharmaceutically acceptable salt thereof to a patient at risk of intramyocardial hemorrhage, cardiac edema, or reperfusion arrhythmias after suffering a myocardial infarction.

In one embodiment, the patient is at risk for intramyocardial hemorrhage or the damage resulting therefrom. In certain embodiments, the patient exhibits one or more risk indicators for intramyocardial hemorrhage. In one embodiment, the one or more risk indicators comprise (i) a diagnosis of ST-segment elevation myocardial infarction (STEMI), (ii) an increase in a marker for myocardial damage, (iii) in vivo imaging evidence of an intramyocardial hemorrhage; (iv) a diagnosis of MVO, no-flow or slow-flow, and (vi) any combination thereof. In certain embodiments, the determining is carried out by in vivo imaging, e.g., by magnetic resonance imaging. In one embodiment, the marker for myocardial damage is a troponin or creatine kinase. In another embodiment, the diagnosis of STEMI is determined by an electrocardiogram (ECG). In another embodiment, the MVO, no-flow or slow flow is determined by x-ray. In certain embodiments, the deferiprone or pharmaceutically acceptable salt thereof is administered in combination with a percutaneous coronary intervention (PCI) or a thrombolytic therapy.

In another embodiment, the deferiprone or pharmaceutically acceptable salt thereof is administered before, during or after the percutaneous coronary intervention (PCI), e.g., coronary angioplasty or insertion of a stent, or a thrombolytic therapy, e.g., administering a thrombolytic agent selected from the group consisting of streptokinase, urokinase, alteplase, recombinant tissue plasminogen activator (rtPA), reteplase, tenecteplase, and any combination thereof.

In certain embodiments of the invention, deferiprone or a pharmaceutically acceptable salt thereof is administered as part of a pharmaceutical composition comprising a pharmaceutically acceptable carrier. In some embodiments, the pharmaceutical composition is an immediate release, sustained release or controlled release pharmaceutical composition.

In certain embodiments of the invention, the patient has suffered at least one episode of myocardial infarction prior to administration of said deferiprone or pharmaceutically acceptable salt thereof.

In some embodiments, the deferiprone or pharmaceutically acceptable salt thereof is administered less than about twenty-four hours, twelve hours, four hours, or two hours after the first episode of myocardial infarction. In certain embodiments, the deferiprone or pharmaceutically acceptable salt thereof is administered within one, two, three, four, five, six, twelve or twenty-four hours of an episode of myocardial infarction.

In some embodiments, the patient experiences angina, dyspnea on exertion, or congestive heart failure prior to administration of said deferiprone or pharmaceutically acceptable salt thereof.

In some embodiments, the deferiprone or pharmaceutically acceptable salt thereof is administered to a patient for a first period of time while the patient is suffering from a myocardial infarction and for a second period of time after which the patient has suffered the myocardial infarction.

Another aspect of the invention is directed to a method of promoting the beneficial remodeling of cardiac tissue in a patient, comprising administering a therapeutically effective amount of deferiprone or a pharmaceutically acceptable salt thereof to a patient before, during or after reperfusion therapy following myocardial ischemia or an acute coronary event in said patient.

Another aspect of the invention is directed to a method of promoting the beneficial remodeling of cardiac tissue following a surgical or catheter-based revascularization procedure, comprising administering a therapeutically effective amount of deferiprone or a pharmaceutically acceptable salt thereof to a patient undergoing the surgical or catheter-based revascularization procedure.

In certain embodiments, the deferiprone or pharmaceutically acceptable salt thereof is administered to the patient for a first period of time prior to and/or during the revascularization procedure and for a second period of time after the patient has completed the revascularization procedure. In some embodiments, the deferiprone or pharmaceutically acceptable salt thereof is administered intravenously to said patient during said first period of time. In some embodiments, the deferiprone or pharmaceutically acceptable salt thereof is administered orally to said patient for said second period of time. In a further embodiment, the second period of time is at least one week or is one week to six months. In certain embodiments, the cardiac tissue is injured by surgery. In one embodiment, the surgery is coronary artery bypass grafting, correction of a congenital heart defect, replacement of a heart valve, or heart transplantation. In another embodiment, there is a hemorrhage in said injured cardiac tissue.

In some embodiments, the deferiprone or pharmaceutically acceptable salt thereof is administered orally or intravenously to said patient. In certain embodiments, the deferiprone or pharmaceutically acceptable salt thereof is administered in one to six doses per day. In certain embodiments, therapeutically effective amount is 1 to 50 mg/kg of deferiprone or equivalent amount of the pharmaceutically acceptable salt thereof administered in one or more oral doses per day up to a maximum of 150 mg/kg/day. In other embodiments, the therapeutically effective amount is 1 to 50 mg/kg/day of deferiprone or equivalent amount of the pharmaceutically acceptable salt thereof in an intravenous pharmaceutical composition administered in one or more intravenous doses per day up to a maximum of 150 mg/kg/day.

In some embodiments of the invention, a second iron chelating agent is administered to the patient. In certain embodiments, the second iron chelating agent is selected from the group consisting of deferoxamine, deferasirox, desferrithiocin, derivatives thereof, e.g., FBS0701, and combinations thereof.

In some embodiments of the invention, an antiplatelet therapy is also administered to the patient. In certain embodiments, the antiplatelet therapy is selected from the group consisting of aspirin, clopidogrel, prasugrel, ticagrelor, ticlopidine, cilostazol, abciximab, eptifibatide, tirofiban, dipyridamole, terutroban, epoprostenol, streptokinase, a plasminogen activator, and combinations thereof.

Another aspect of the invention is directed to a method of selecting a patient for treatment of a myocardial hemorrhage with deferiprone or a pharmaceutically acceptable salt thereof, comprising determining whether there is a myocardial hemorrhage in the patient after a myocardial infarction.

Another aspect of the invention is directed to a method of treating or ameliorating a myocardial hemorrhage in a patient, comprising (a) determining whether there is a myocardial hemorrhage in the patient after a myocardial infarction, and (b) administering a therapeutically effective amount of deferiprone, or a pharmaceutically acceptable salt thereof, to said patient if it is determined that there is a hemorrhage at the place of the infarct.

In certain embodiments, the determining is carried out by in vivo imaging. In some embodiments, the in vivo imaging is by magnetic resonance imaging.

A previous invention demonstrated that deferiprone could prevent or treat heart failure in patients with transfusional iron overload (U.S. Pat. No. 7,049,328 B2), a condition that takes a decade or more of transfusions to develop. It was unexpected that in subjects without iron overload, such as those who are not transfused, might benefit from deferiprone after an acute myocardial event such as a heart attack or other components of an acute coronary syndrome, because there is no generalized build up of iron in the body and no measurable increase of iron deposition in the heart, as in the case of iron overload patients. According to the methods of the present invention, patients for whom there is no generalized iron overload are treated with deferiprone or a pharmaceutically acceptable salt thereof.

In another embodiment, the invention provides a method of limiting reperfusion injury of cardiac tissue following a surgical or catheter-based revascularization procedure, comprising administering a therapeutically effective amount of deferiprone, or a pharmaceutically acceptable salt thereof, to a patient for a first period of time prior to and/or during which the patient is undergoing the revascularization procedure and for a second period of time after the patient has completed the revascularization procedure.

In another embodiment, the invention provides a method of limiting reperfusion injury and/or promoting the beneficial remodeling of cardiac tissue following a surgical or catheter-based revascularization procedure, comprising administering a therapeutically effective amount of deferiprone, or a pharmaceutically acceptable salt thereof, to a patient for a first period of time prior to and/or during which the patient is undergoing the revascularization procedure and for a second period of time after the patient has completed the revascularization procedure.

The invention also provides a method of selecting a patient for treatment with deferiprone, or a pharmaceutically acceptable salt thereof, to treat myocardial infarction, comprising determining whether there is a hemorrhage at the place of the infarct.

The invention also provides a method of treating or ameliorating myocardial infarction in a patient, comprising (a) determining whether there is a hemorrhage at the place of the infarct, and (b) administering a therapeutically effective amount of deferiprone, or a pharmaceutically acceptable salt thereof, to said patient if it is determined that there is a hemorrhage at the place of the infarct.

The invention also provides deferiprone for use in a therapeutic method according to the present invention. The invention also provides use of deferiprone in the manufacture of a medicament for use in a therapeutic method according to the present invention.

### BRIEF DESCRIPTION OF THE DRAWINGS/FIGURES

FIG. 1: Longitudinal changes in Edema, Hemorrhage and MVO. T2 and T2* maps are shown along with early contrast enhanced (CE) images at various time points post-AMI in a representative animal. Day 2: T2 elevation usually associated with edema was not apparent in the infarct zone (39.2 ms vs. 39.1 ms control) but was slightly elevated in the peripheral areas; diastolic wall thickness (DWT) was also increased by 34% suggesting edematous swelling. Arrows indicate focal signal-void regions or T2* abnormalities (18.5 ms vs. 34.2 ms control) within the MVO as delineated by the CE image. Week 1: T2 was elevated (arrows) in most of the infarct (51.1 ms) with reduced sub-endocardial T2* (15.8 ms) indicative of diffuse hemorrhagic byproducts (arrows). Week 4: T2 was still elevated (50 ms) while normalization of T2* (35 ms) coincided with resolution of MVO.

FIG. 2: Quantitative fluctuations in MRI parameters after AMI. Plots (a) and (b) show longitudinal fluctuations in T2 and T2* in infarct zone compared to remote myocardium averaged over all animals; error bars represent standard error. Plots (c) and (d) show evolution of infarct and MVO size. Plot (e) shows regional alterations in DWT while (f) indicates global left ventricular function as represented by ejection fraction (EF). Day 0 represents values from healthy controls. MRI scans: day 0 (N=10), day 2 (N=8), week 1 (N=5), week 2 (N=8), week 4 (N=5) and week 6 (N=4). †p<0.05, compared to control values; §p<0.05, compared to the previous time point.

FIG. 3: Short axis slices from MRI (a-c) are compared to corresponding Hematoxylin and Eosin (HE) stained histology slide (d) in a porcine heart at day 2. Images (e)-(j) are magnified versions of regions 1-4 (squares) indicated in (d). Image (e) 40×, shows remote zone showing viable myocytes. In image (f) 4×, the arrow points to hemorrhagic core within MVO which corresponded with T2* signal void in (c). Image (g) 100×, is a magnified version of (f) showing intact red blood cells (arrowheads) along with inflammatory cells. Image (h) 100×, shows widespread necrosis in the infarct core (arrowheads). Image (i) 10×, shows edema indicated by wide interstitium (arrowheads). Image (j) 40×, shows Von Kossa (VK) staining, which indicates calcium deposition in the infarct periphery (arrowheads). HE: Hematoxylin and Eosin; VK: Von Kossa (VK).

FIG. 4: Plots demonstrate evolution of resting T2 (blue), stress T2 (red) and changeΔT2 (black) post-AMI in porcine hearts. In the infarct zone (a,b), rest and stress T2's were both significantly elevated by more than 40% compared to day 0 (control) values at all the time points beyond week 1 (p<0.005), indicative of edema. Having no significant difference between rest and stress values suggested non-salvageable myocardium. In the remote zone (c,d), rest T2 was subtly elevated by ˜7% at week 1 (p=0.055) and week 2 (p=0.058) which suggested either edema or hyperemia. These results were accompanied by a simultaneous suppression of stress response, i.e. stress T2 was depressed at weeks 1-2, indicative of vasodilatory dysfunction.

FIG. 5: Delayed hyperenhanced (DHE) images from representative animals demonstrated differences between the 90 min and 45 min occlusion groups. Basal slice (blue localizer) was un-infarcted and was utilized for remote myocardium/zone assessment. In the apical slice (red localizer), MVO was apparent at day 2 in the large 90 min infarct, identified as a region of hypoenhancement within the hyperenhanced myocardium (white arrows) that resolved by week 4. On the other hand, the 45 min animal demonstrated small, non-transmural and heterogeneous infarct. LAX—Long Axis; SAX—Short Axis.

FIG. 6: In the 90 min occlusion, T2-(TE=88 ms) and T2*-weighted (TE=15 ms) images demonstrated edema (bright signal) and hemorrhage (signal void), respectively, that corresponded with the transmural infarction and MVO (blue arrows). The 45 min occlusion showed bright edema signal on T2-weighted image with no apparent signal voids on the T2*-weighted image. T2 and T2* maps (color bar in ms) showed the quantitative aspect of myocardial tissue characterization. White arrows in the lateral regions on T2* maps indicate susceptibility artifacts arising from the heart-lung interface and cardiac veins.

FIG. 7: Cumulative time course of T2 and T2* parameters post-AMI pooled across all animals in the 90 min (a-c) and 45 min (d-f) groups; error bars show standard error and day 0 indicates control MRI. Plots (a), (d) represent fluctuations in T2 within infarct zone while plots (b), (e) represent remote zone under rest and stress conditions. Plots (c), (f) demonstrate T2* alterations in infarct and remote zones. Shaded area in plots (b) and (e) indicates impaired vasodilatory function while that plot (c) shows depressed T2* indicative of hemorrhage. *p<0.05 compared to control; †p<0.05 compared to rest.

FIG. 8: Cumulative time course of ejection fraction (EF) and end-diastolic volume (EDV) post-AMI compared in the 90 and 45 min groups; error bars show standard error. *p=0.05 compared to control (day 0). Depressed EF and larger EDV at 6 weeks indicates greater remodeling in the 90 min group.

FIG. 9: Short axis slices from patients who underwent MRI exam at day 2 post-PCI. The distinct patterns of myocardial damage were shown by the delayed enhancement images. Signal void region (arrows) in the T2* image shows myocardial hemorrhage within the infarcted territory.

FIG. 10: Representative short axis T2, T2* and delayed-enhanced (DHE) images from a patient who underwent MRI exam at day 2, week 4 and month 6 post-PCI. The indicated values show progression of T2 and T2* measurements over time in the infarct and remote myocardium.

FIG. 11: Top panel: Evolution of T2 and T2* in patients. Elevated T2 in the infarct zone reflects edema while depressed T2* indicates hemorrhage. In general, edema was resolved by month 6 while hemorrhage was resolved by week 2-4. Bottom panel: The plots demonstrate T2 alterations in infarcted and remote myocardium in two sub-groups of patients: with and without hemorrhage. At day 2 in the infarct zone, T2 was lower in patients with hemorrhage showing that edema and hemorrhage had counteracting effects on T2 values. At day 2 in the remote zone, T2 was higher in the patients with hemorrhage, which was indicative of edema or hyperemia in distal un-infarcted myocardium.

FIG. 12: Top panel: Pig hearts treated with an intracoronary injection of collagenase beyond the second diagonal branch of the left anterior descending artery (LAD) (inset in 600 mcg image) after a brief ischemic episode of 8 min. For a dose of >800 mcg, collagenase resulted in hemorrhage (reddish areas) as is apparent on the explanted hearts; amount of hemorrhage increased with dose. Bottom panel: Although hemorrhage appeared to be epicardial, staining revealed moderate to severe blood spill in the myocardium as well. Hematoxylin and eosin stains from the right and left ventricle (RV, LV) demonstrated widespread areas of red blood cells dispersed throughout the myocardium. No infarction was observed.

FIG. 13: Left panel: T2-, T2*-weighted and DHE short-axis images from an animal subjected to a 45 min LAD occlusion followed by 1000 mcg injection of collagenase during reperfusion. Signal void on T2* image indicated a hemorrhagic core (red arrow) that corresponded with an MVO (hypoenhanced region within hyperenhanced rim of gadolinium) on the DHE image. Appearance of MVO was unlike untreated 45 min infarcts seen in FIG. 5. These results show an interaction between hemorrhage, MVO and infarction. Right panel: Apical MVO (red arrow) seen on a long-axis view of the DHE image.

FIG. 14: Short axis images from a representative animal subjected to 90 min LAD occlusion and treated with iron chelator deferiprone (DFP). Hemorrhage, as indicated by T2* image (red arrow), was observed only on day 2 but resolved by week 1. Edema also substantially subsided at week 4. Persistent MVO was seen on day 2 that was partially resolved by week 1. The results from this example are in contrast to the untreated 90 min group shown in FIG. 1.

FIG. 15: Cumulative time course of T2, T2* and cardiac function parameters post-AMI pooled across all animals (N=2) in the 90 min infarct group treated with deferiprone; error bars show standard error and day 0 indicates control MRI. Shaded area on T2* images indicates that hemorrhage was only observed on day 2, which later resolved. Edema was substantially subsided beyond week 2 (shaded area). In the remote myocardium, T2 results show impaired vasodilatory function only at day 2 (shaded area), resolving thereafter, although not to control levels. Ejection fraction (EF) reduced gradually probably as a result of the severe 90 min occlusion, although not abruptly from day 2 like the untreated 90 min animals (see FIG. 2). Relatively unchanged end-diastolic and end-systolic volumes (EDV, ESV) indicated less adverse remodeling at 4 weeks.

FIG. 16: Short axis images from representative animals subjected to 90 minutes LAD occlusion without (top panel) and with (bottom panel) treatment of iron chelator deferiprone (DFP). In the DFP treated group, hemorrhage (as indicated by T2* image (red arrows)) was observed only on day 2 and resolved by week 1. In both the treated and untreated groups, microvascular obstruction (MVO) was seen on day 2 and was partially resolved by week 1.

FIG. 17: Cumulative time course of T2 and T2* parameters post-AMI pooled across all animals (N=2) in 90 minute infarct—untreated and treated with deferiprone (DFP); error bars show standard error and day 0 indicates control MRI scans in healthy animals.

### DETAILED DESCRIPTION OF THE INVENTION

### List of Abbreviations

PCI: percutaneous coronary intervention
STEMI: ST-segment elevation myocardial infarction
ECG: electrocardiogram
RI: reperfusion injury
MVO: ischemia-induced microvascular obstruction

LV/RV: left/right ventricle or left/right ventricular

ACE: angiotensin-converting enzyme
(A)MI: (acute) myocardial infarction
RISK: reperfusion injury salvage kinase pathway
PTP: mitochondrial permeability transition pore
ROS: reactive oxygen species
MRI: magnetic resonance imaging
DHEL delayed hyperenhancement MRI
AAR: area-at-risk
DWT: diastolic wall thickness
EF: ejection fraction
HE: hematoxylin and eosin

### VK: Von Kossa

EDV: end-diastolic volume
LAD: left anterior descending artery
DFP: deferiprone
EDV/ESV: end-diastolic and end-systolic volumes
BOLD: blood-oxygen-level-dependent imaging
MLLSR: modified Look-Locker sequence with saturation recovery
PR: picrosirius red
PB: Perl's prussian blue
CE: contrast enhanced
CMR: cardiovascular magnetic resonance
TIMI: thrombolysis in myocardial infarction

### Deferiprone

As used herein deferiprone (or “DFP”) refers to deferiprone or a pharmaceutically acceptable salt thereof. Salts of deferiprone include pharmaceutically acceptable salts, especially salts with bases, such as appropriate alkali metal or alkaline earth metal salts, e.g., sodium, potassium or magnesium salts, pharmaceutically acceptable transition metal salts, such as zinc salts, or salts with organic amines, such as cyclic amines, such as mono-, di- or tri-lower alkylamines, such as hydroxy-lower alkylamines, e.g. mono-, di- or trihydroxy-lower alkylamines, hydroxy-lower alkyl-lower alkylamines or polyhydroxy-lower alkylamines. Cyclic amines are, e.g. morpholine, thiomorpholine, piperidine or pyrrolidine. Suitable mono-lower alkylamines are, e.g. ethyl- and tert-butylamine; di-lower alkylamines are, e.g., diethyl- and diisopropylamine; and tri-lower alkylamines are, e.g. trimethyl- and triethylamine. Appropriate hydroxy-lower alkylamines are, e.g. mono-, di- and triethanolamine; hydroxy-lower alkyl-lower alkylamines are, e.g. N,N-dimethylamino- and N,N-diethylaminoethanol; a suitable polyhydroxy-lower alkylamine is, e.g. glucosamine.

### Methods of Using Deferiprone

Terms such as “treating” or “treatment” or “to treat” or “ameliorating” or “alleviating” or “to alleviate” may refer to both 1) therapeutic measures that cure, slow down, lessen symptoms of, reverse, and/or halt progression of a diagnosed pathologic condition or disorder and 2) prophylactic or preventative measures that prevent and/or slow the development of a targeted pathologic condition or disorder. Thus those in need of treatment include those already with the disorder; those prone to have the disorder; and those in whom the disorder is to be prevented. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. In certain embodiments, the patient is a human. In certain embodiments, the patient does not suffer from generalized iron overload or a cardiac condition associated with transfusional iron overload e.g., due to blood transfusion treatments.

By “therapeutically effective dose or amount” or “effective amount” is intended an amount of deferiprone that when administered brings about a positive therapeutic response with respect to treatment of a patient with a disease to be treated.

### Myocardial Ischemia or Acute Coronary Event

Myocardial ischemia is an imbalance between myocardial oxygen supply and demand. If left untreated, myocardial ischemia can result in, e.g., angina pectoris, myocardial stunning, myocardial hibernation, ischemic preconditioning, postconditioning, or under the most severe instances, acute coronary syndrome and/or myocardial infarction.

As used herein an acute coronary event may include, e.g., an acute coronary syndrome (ACS), e.g., an acute myocardial infarction or a ST-segment elevation myocardial infarction (STEMI).

Acute myocardial infarction occurs due to cessation of blood flow into the heart muscle, thereby resulting in irreversible necrosis in the region supplied by the concerned coronary artery (5). The extent of tissue injury is proportional to the duration of occlusion, with myocardial damage following a wavefront phenomenon of ischemic cell death (6)—subendocardial to transmural. In STEMI patients treated with PCI, prolonged times between symptom onset (chest pain, etc.) and reperfusion (>4 hrs) are associated with impaired ST-segment resolution, larger infarcts and higher mortality (7, 8). Based on a recent study in 5000+ STEMI patients (9), for symptom-to-reperfusion times, mortality was <3 hrs: 3.7%; 3-5 hrs: 4.2%; and >5 hours: 6.5%, while for door-to-reperfusion times, it was <60 min: 3.2%; 60-90 min: 4.0%; 90-120 min: 4.6%; and >120 min: 5.3%. Strategies to minimize delays in PCI are critical for angioplasty centers.

In one embodiment, the invention provides a method for treating or ameliorating myocardial ischemia or an acute coronary event, comprising administering a therapeutically effective amount of deferiprone or a pharmaceutically acceptable salt thereof to a patient in need thereof.

In one embodiment, the invention provides a method for treating or ameliorating myocardial ischemia or an acute coronary event, e.g., an acute myocardial infarction or a ST-segment elevation myocardial infarction (STEMI), comprising administering a therapeutically effective amount of deferiprone or a pharmaceutically acceptable salt thereof to a patient for a period of time after which the patient has suffered the myocardial ischemia, an acute coronary event, e.g., acute myocardial infarction. In one embodiment, the patient has experienced a myocardial infarction and did not receive treatment with deferiprone, or a pharmaceutically acceptable salt thereof, during the acute phase. Administration of deferiprone, or a pharmaceutically acceptable salt thereof after the acute phase may enhance recovery from the myocardial infarction.

In one embodiment, the deferiprone or pharmaceutically acceptable salt thereof is administered to a patient who presents with an acute coronary syndrome or event and is suspected of having suffered a myocardial infarction, for example, by paramedics. In certain embodiments, the patient may exhibit one or more symptoms of myocardial infarction including neck and shoulder pain, chest pain, pain in the left arm, abdominal pain, nausea, vomiting, fatigue, and shortness of breath.

In another embodiment, the deferiprone or pharmaceutically acceptable salt thereof is administered to the patient who has been diagnosed as having suffered a myocardial infarction, for example, by an emergency room physician or doctor who may have carried out or is advised of the results from a cardiac catheterization procedure.

In order to limit the damage resulting from myocardial infarction, in certain embodiments, it is preferred that the patient be treated with deferiprone or a pharmaceutically acceptable salt thereof, and optionally with other agents known to be useful for treating myocardial infarction, as soon as possible following diagnosis. In certain embodiments, intravenous administration is preferred, e.g., in situations where it is desirable to provide for therapeutic blood levels of deferiprone in the shortest period of time and/or when a patient is unable to swallow or is unconscious.

In some embodiments, the deferiprone or a pharmaceutically acceptable salt thereof is administered for a first period of time while the patient is suffering from the myocardial ischemia or an acute coronary event and for a second period of time after the patient has suffered the myocardial ischemia or an acute coronary event.

In a further embodiment, the deferiprone or pharmaceutically acceptable salt thereof is administered intravenously to the patient during the first period of time when the patient is suffering from myocardial ischemia or an acute coronary event. In another embodiment, the therapeutically effective amount of deferiprone is 1 to 150 mg/kg/day, or an equivalent amount of the pharmaceutically acceptable salt thereof, in an intravenous pharmaceutical composition. In one embodiment, deferiprone or pharmaceutically acceptable salt thereof can be administered intravenously for up to three hours or less; up to two hours or less; or up to one hour or less. In another embodiment, the continuous intravenous administration is at least 15, 30, or 45 minutes and up to 1, 2, or 3 hours. In another embodiment, the administration does not exceed serum concentration levels of deferiprone or pharmaceutically acceptable salt thereof of 50 micromolar or more throughout a dosing interval.

In another embodiment, during the second period of time after the acute phase, deferiprone or a pharmaceutically acceptable salt thereof is administered orally to said patient to enhance recovery from the myocardial infarction. A therapeutically effective oral amount is 1 to 150 mg/kg of deferiprone an in oral pharmaceutical composition, or an equivalent amount of the pharmaceutically acceptable salt thereof. The second period of time of administration can continue for at least one week, or at least one month.

### Reperfusion

As use herein, “reperfusion” refers to return of bloodflow to or perfusion of an ischemic tissue or organ, e.g., ischemic myocardium. Following myocardial ischemia or an acute coronary event, early restoration of coronary perfusion to the ischemic myocardium is currently the most effective strategy to limit infarct size and ventricular arrhythmias, and thereby prevent cardiac failure and death (2).

In certain embodiments, reperfusion is achieved with percutaneous coronary intervention (PCI) or thrombolytic therapy. Standard practice in North American hospitals for patients presenting an ST-segment elevation myocardial infarction (STEMI) by electrocardiogram (ECG) is to directly refer for PCI since the benefits of therapy are maximized when patients are treated early (3, 4). Reperfusion therapy further accelerates the inflammatory and healing process, especially in the case of larger infarcts. In certain embodiments, the subject of the methods disclosed herein is treated with a therapy to promote reperfusion, e.g., a thrombolytic therapy or PCI, before, during or after administration of deferiprone.

Reperfusion injury (RI) is the tissue damage caused when blood supply returns to the tissue after a period of ischemia or lack of oxygen. The absence of oxygen and nutrients from blood during the ischemic period creates a condition in which the restoration of circulation results in inflammation and oxidative damage through the induction of oxidative stress. While reperfusion is favorable in terms of myocardial salvage, it can result in additional cardiac damage rivaling that of the initial event (10). RI has been associated with worsening or expansion of the prior ischemic damage resulting in microvascular dysfunction arising from endothelial cell damage, stunning, reperfusion arrhythmias, and further myocyte death. In one embodiment, the methods disclosed herein are directed to treating or reducing the risk of reperfusion injury in a subject. In certain embodiments of the invention, the reperfusion injury is a myocardial reperfusion injury. In certain embodiments, the injury also exhibits any one or more of the following pathologic processes: intramyocardial hemorrhage, cardiac edema, arrhythmias, ischemic damage, apoptosis, stunning and additional irreversible injury in addition to the ischemic injury.

Intracellular and interstitial edema is a consistent feature of RI in acute myocardial infarction (AMI) arising from a local inflammatory reaction (11). In addition, a phenomenon called ‘no-reflow’ is often encountered, which is typically caused by ischemia-induced microvascular obstruction (MVO) and injury and has been correlated with adverse left ventricular (LV) remodeling and poor patient outcome (12). Furthermore, reperfusion coupled with a severe initial ischemic insult may also result in intramyocardial hemorrhage (13), which in association with MVO is believed to be an independent predictor of adverse remodeling (14). In some patients, vascular compromise manifests itself during the procedure as an abrupt decrease in epicardial blood flow—Thrombolysis In Myocardial Infarction (TIMI) grade 0 to 1—i.e., “no-reflow” or “slow-reflow”.

Ischemia-induced microvascular obstruction (MVO) in the heart can include endothelial cell swelling, and endothelial protrusion by cell swelling together with neutrophils, red blood cells, and platelets can cause capillary obstruction (see, e.g., Kloner et al., J Clin Invest 54:1496-1508 (1974)). MVO is independently associated with adverse ventricular remodeling and patient prognosis. Several techniques (e.g., coronary angiography, myocardial contrast echocardiography, cardiovascular magnetic resonance imaging, and electrocardiography) measuring slightly different biological and functional parameters are used clinically and experimentally to detect MVO. Sebastiaan et al., J Am Coll Cardiol 55(16):1649-1660 (2010).

In a recent study, Eitel et al. showed that 35% of 346 patients who were determined to have STEMI had indications of hemorrhage as shown by MRI (Circ. Cardiovasc. Imaging 4:354-362 (2011)).

In one embodiment, the invention is directed to treating a patient at risk for intramyocardial hemorrhage or the damage resulting therefrom, at risk for cardiac edema or the damage resulting therefrom, at risk for reperfusion arrhythmias or the damage resulting therefrom, at risk for other ischemic damage to the heart, or at risk for any combination thereof. In one embodiment, the patient at risk has suffered an acute myocardial infarction or a ST-segment elevation myocardial infarction (STEMI) and has been given reperfusion therapy.

In another embodiment, the patient at risk for intramyocardial hemorrhage or the damage resulting therefrom is diagnosed with or determined to have one or more of the following risk indicators: (i) ST-segment elevation myocardial infarction (STEMI), e.g., determined by ECG; (ii) an increase in one or more markers for myocardial damage, e.g., increased creatine kinase and/or troponin levels (e.g., cardiac troponin I and T), e.g., determined by a troponin test; (iii) microvascular obstruction and/or no-reflow or slow-reflow, e.g., determined by x-ray (e.g., a Pre-PCI TIMI flow value of 0 or 1); and (iv) imaging evidence of an intramyocardial hemorrhage, e.g., determined by in vivo imaging (e.g., MRI or CMR). See, e.g., Ganame et al., European Heart Journal 30:1440-1449 (2009); and Mather et al., Heart 97:453-459 (2011).

In one embodiment, deferiprone or a pharmaceutically acceptable salt thereof is administered to the patient at risk for intramyocardial hemorrhage or the damage resulting therefrom. In another embodiment, the methods disclosed herein are directed to treating or reducing the risk of intramyocardial hemorrhage or the damage resulting therefrom in a patient, e.g., a patient diagnosed with or determined to have one or more risk indicators for intramyocardial hemorrhage disclosed herein.

In one embodiment, deferiprone or a pharmaceutically acceptable salt thereof is administered to a patient at risk for intramyocardial hemorrhage or the damage resulting therefrom, wherein the patient has been diagnosed with or determined to have ST-segment elevation myocardial infarction (STEMI), e.g., determined by ECG (e.g., performed at the time of initial evaluation by a health care provider, e.g., in the ambulance). In a further embodiment, the methods disclosed herein are directed to treating or reducing the risk of intramyocardial hemorrhage or the damage resulting therefrom in a patient diagnosed with or determined to have ST-segment elevation myocardial infarction (STEMI).

In one embodiment, deferiprone or a pharmaceutically acceptable salt thereof is administered to a patient at risk for intramyocardial hemorrhage or the damage resulting therefrom, wherein the patient has been diagnosed with or determined to have an increase in a marker for myocardial damage, e.g., elevated cardiac enzyme level indicative of necrosed cardiac muscle (e.g., creatine kinase) and/or troponin levels (e.g., cardiac troponin I and T) (e.g., tested in the emergency room). In a further embodiment, the methods disclosed herein are directed to treating or reducing the risk of intramyocardial hemorrhage or the damage resulting therefrom in a patient diagnosed with or determined to have an increase in a marker for myocardial damage.

In one embodiment, deferiprone or a pharmaceutically acceptable salt thereof is administered to the patient at risk for intramyocardial hemorrhage or the damage resulting therefrom, wherein a patient has been diagnosed with or determined to have microvascular obstruction and/or no-reflow or slow-reflow following revascularization (e.g., pre-PCI TIMI flow values of 0 or 1), e.g., determined by x-ray (e.g., assessed during revascularization). In a further embodiment, the methods disclosed herein are directed to treating or reducing the risk of intramyocardial hemorrhage or the damage resulting therefrom in a patient diagnosed with or determined to have microvascular obstruction and/or no-reflow or slow-reflow following revascularization.

In one embodiment, deferiprone or a pharmaceutically acceptable salt thereof is administered to a patient at risk for intramyocardial hemorrhage or the damage resulting therefrom, wherein the patient has been diagnosed with or determined to have imaging evidence of an intramyocardial hemorrhage, e.g., determined by in vivo imaging, e.g., MRI or CMR (e.g., assessed after revascularization). In a further embodiment, the methods disclosed herein are directed to treating or reducing the risk of intramyocardial hemorrhage or the damage resulting therefrom in a patient diagnosed with or determined to have imaging evidence of an intramyocardial hemorrhage.

In another embodiment, the patient at risk for myocardial edema or the damage resulting therefrom is diagnosed with or determined to have one or more of the following risk indicators: (i) ST-segment elevation myocardial infarction (STEMI), e.g., determined by ECG; (ii) an increase in one or more markers for myocardial damage, e.g., increased creatine kinase and/or troponin levels (e.g., cardiac troponin I and T), e.g., determined by a troponin test; (iii) microvascular obstruction and/or no-reflow or slow-reflow, e.g., determined by x-ray (e.g., a Pre-PCI TIMI flow value of 0 or 1); and (iv) imaging evidence of an myocardial edema, e.g., determined by in vivo imaging (e.g., MRI or CMR)

Clinical findings suggest that patients with durations of ischemia >4 hrs are at significantly high risk of experiencing major adverse cardiovascular events such as re-infarction, repeat revascularization, heart failure and death; mortality appears to be highest with hemorrhagic infarcts. In another embodiment, the patient at risk has experienced a long duration between symptoms of myocardial infarction (e.g., neck and shoulder pain, chest pain, pain in the left arm, abdominal pain, nausea, vomiting, fatigue, and shortness of breath) and the start of treatment for myocardial infarction (e.g., a duration of greater than about 4 hours). In certain embodiments, a patient at risk for a intramyocardial hemorrhage or the damage resulting therefrom, a cardiac edema or the damage resulting therefrom, a reperfusion arrhythmias or the damage resulting therefrom, other ischemic damage to the heart, or any combination thereof is assessed to determine the severity of the ischemic damage or size of infarction. In one embodiment, a patient is diagnosed or determined to have a large infarction size, and therefore determined to be at greater risk for MVO and/or hemorrhage, edema or arrhythmias.

Although occurrence of myocardial hemorrhage following AMI has been known for many years from human autopsy studies and preclinical tissue specimens (24-26), this feature had been neglected, in the past, due to lack of sensitive imaging techniques for in vivo detection. The in vivo identification of hemorrhage is a relatively new development, especially in humans, and furthermore, there has been renewed interest in reperfusion hemorrhage due to its adverse consequences presented in the clinic. Hemorrhage-sensitive MRI sequences (T2, T2*) have been particularly instrumental in this regard (27-29) and have also made it possible to study the long-term consequences of hemorrhage in clinical practice. A recent study by Mather et al. (30) demonstrated that reperfusion hemorrhage was associated with large infarct size, reduced salvage, greater MVO and lower ejection fraction; it was also the strongest independent predictor of adverse LV remodeling, with an increased risk of arrhythmia and predictive power even greater than MVO. Recent studies have indicated that clinical presentation of MVO and hemorrhage can be as high as 50% and 25%, respectively (14, 30, 31). Although clinical studies can establish the relationship between hemorrhage and outcomes, many of its aspects in AMI are yet to be understood.

In one embodiment, the patient treated by the methods disclosed herein has a MVO or is at risk for MVO. In another embodiment, the patient treated by the methods disclosed herein has a MVO and a myocardial hemorrhage or is at risk for MVO and a myocardial hemorrhage.

The presentation of intramyocardial hemorrhage as a consequence of reperfusion injury in AMI has been documented in both humans (14, 33) and animal models (25, 26, 34, 35). Not only oxidative stress, but also calcium overload, pH fluctuation, increased inflammation, and mitochondrial damage are the predominant components of reperfusion injury that result in cellular and vascular damage. Reperfusion appears to be a prerequisite for tissue hemorrhage, and greater initial ischemic insult durations have been attributed to greater degrees of hemorrhage (25, 26, 36). Hemorrhage is confined within the area of necrosis and is most likely caused by leakage of blood from damaged microvasculature in the ischemic territory. It has been demonstrated in experimental models that microvascular injury lags behind myocardial cell injury (37). Furthermore, hemorrhage has been found to be associated with MVO (32, 38); however, knowledge of the relationship between the two is lacking. It is not known whether MVO causes endothelial damage resulting in blood leakage, making hemorrhage simply a marker of severity, or whether hemorrhage causes myocardial swelling and compression of vessels leading to or worsening an MVO. Patients with hemorrhagic infarcts appear to be at high risk, with poor long-term outcomes (14, 47). The changing appearance of tissue hemorrhage has been extensively studied in the case of brain hemorrhage (39). Hemorrhage undergoes a dynamic transformation process as it ages in the tissue: 1) hyperacute phase (<24 h)—intracellular oxyhemoglobin (ferrous); 2) acute (<3 days)—intracellular deoxyhemoglobin (ferrous); 3) early subacute (>3 days)—intracellular methemoglobin (ferric); 4) late subacute (>7 days)—extracellular methemoglobin (ferric); and 5) chronic (>14 days)—extracellular ferritin and hemosiderin (ferric). Degradation products of hemoglobin have been associated with increased brain edema, neuronal damage and neurological defects (40, 41).

In certain embodiments of the invention, the reperfusion injury is a myocardial reperfusion injury, e.g., a myocardial reperfusion injury which also exhibits any one or more of the following pathologic processes: intramyocardial hemorrhage, cardiac edema, arrhythmias, ischemic damage, apoptosis, stunning and additional irreversible injury in addition to the ischemic injury.

In certain embodiments, the intramyocardial hemorrhage or the damage resulting therefrom, the cardiac edema or the damage resulting therefrom, the arrhythmias or the damage resulting therefrom, or the ischemic damage to the heart is the result of a reperfusion therapy, e.g., a thrombolytic therapy or PCI.

Hemorrhage is a source of iron toxicity and a mediator of inflammation, directly contributing to adverse LV remodeling in the setting of AMI; tissue characterization by quantitative-MRI can be used to demonstrate the role of hemorrhage and to ultimately guide therapeutic decision making and monitor treatment response.

Left ventricular (LV) remodeling following acute myocardial infarction (AMI) is associated with significant morbidity, ultimately leading to cardiovascular dysfunction, disability and death. The current inventors have found that prolonged iron chelation administration following myocardial infarction improves peri-infarct inflammation and remodeling.

As discussed herein, reperfusion injuries, e.g., myocardial hemorrhage and/or edema frequently occur after reperfusion of acute myocardial infarction. In one embodiment of the invention, there is a hemorrhage in the area of the infarct. The presence of a hemorrhage in the area of the infarct may be identified by an imaging technique as described in more detail herein. In certain embodiments, deferiprone, or a pharmaceutically acceptable salt thereof, is administered before, during or after a reperfusion therapy to prevent or treat a myocardial hemorrhage and/or edema.

In certain embodiments, the invention provides a method of reducing the risk of intramyocardial hemorrhage, cardiac edema, reperfusion arrhythmias, and ischemic damage; treating, preventing or ameliorating myocardial ischemia, an acute coronary event or reperfusion injury; and promoting the revascularization and beneficial remodeling of cardiac tissue, comprising administering a therapeutically effective amount of deferiprone or a pharmaceutically acceptable salt thereof to a patient in need thereof. In certain embodiments, deferiprone may be combined with a pharmaceutical carrier to produce a single dosage form which will vary depending upon the patient's weight and the particular mode of administration. The composition may be administered as a single dose, multiple doses or over an established period of time, e.g., in a parenteral infusion. Dosage regimens also may be adjusted to provide the optimum desired response (e.g., a therapeutic or prophylactic response).

The invention also provides a method of treating or limiting reperfusion injury and promoting the beneficial remodeling of cardiac tissue following myocardial ischemia, an acute coronary event, or a surgical or catheter-based revascularization procedure. In certain embodiments, the method comprises administering a therapeutically effective amount of deferiprone, or a pharmaceutically acceptable salt thereof, to a patient for a first period of time prior to and/or during which the patient is undergoing the revascularization procedure and for a second period of time after the patient has completed the revascularization procedure. In one embodiment, the deferiprone or pharmaceutically acceptable salt thereof is administered intravenously to said patient during said first period of time. In another embodiment deferiprone, or a pharmaceutically acceptable salt thereof, is administered orally to said patient during said second period of time.

In another embodiment, a hemorrhage is present in the injured cardiac tissue. The presence of a hemorrhage in the area of the infarct may be identified by an imaging technique as described in more detail elsewhere herein.

In one embodiment, the patient at risk for myocardial reperfusion injuries or other injury associated with myocardial infarction treatment has previously suffered at least one episode of myocardial infarction. In another embodiment, the patient at risk for myocardial reperfusion injuries or other injury associated with myocardial infarction treatment experiences or has experienced angina, dyspnea on exertion, congestive heart failure, cardiovascular disease, atherosclerosis, high cholesterol, high blood pressure, smokes tobacco, has a family history of coronary heart disease at a young age, or is diabetic.

In one embodiment, the cardiac tissue is injured by surgery, for example, by coronary artery bypass grafting, correction of a congenital heart defect, replacement of a heart valve, or heart transplantation.

In another embodiment the cardiac tissue injury, e.g., ischemia, is the result of reperfusion after percutaneous coronary intervention, such as coronary angioplasty. One example of coronary angioplasty is balloon angioplasty, which is used for example to widen a partially occluded coronary artery. In another embodiment, the cardiac tissue injury, e.g., ischemia, is the result of insertion of a stent into a partially occluded coronary artery.

In another embodiment, the cardiac tissue injury, e.g., ischemia, is the result of myocardial infarction. In this embodiment, administration of deferiprone, or a pharmaceutically acceptable salt thereof will promote revascularization and beneficial remodeling of the heart. Adverse remodeling of the heart occurs after myocardial infarction and includes a cascade of biochemical signaling changes that induce dilatation, hypertrophy, and the formation of a collagen scar. Ventricular remodeling may continue for weeks or months until the distending forces are counterbalanced by the tensile strength of the collagen scar. This balance is determined by the size, location, and transmurality of the infarct, the extent of myocardial stenting, the patency of the infarct-related artery or arteries, and local tropic factors.

In one embodiment, the administration of deferiprone, or a pharmaceutically acceptable salt thereof will promote beneficial remodeling of the heart. Administration of deferiprone or pharmaceutically acceptable salt thereof will promote beneficial remodeling by one or more of: reducing ischemia-induced microvascular obstruction (MVO), neutralizing hemorrhagic byproducts; or indirectly reducing edema. In one embodiment, deferiprone or a pharmaceutically acceptable salt thereof is administered prior to said percutaneous coronary intervention. In another embodiment, the deferiprone or pharmaceutically acceptable salt thereof is administered prior to, during and after said percutaneous coronary intervention. In another embodiment, the deferiprone or pharmaceutically acceptable salt thereof is administered after said percutaneous coronary intervention. It is expected that administration of deferiprone or a pharmaceutically acceptable salt thereof before, during and/or after an angioplasty procedure will ameliorate the extent of myocardial tissue damage, and that the myocardial tissue will exhibit beneficial remodeling and, therefore, recover more quickly.

In certain embodiments, when administered before or during the percutaneous coronary intervention, the deferiprone or pharmaceutically acceptable salt thereof may be administered by intravenous means. In certain embodiments, when administered before or after the percutaneous coronary intervention, the deferiprone, or an equivalent amount of the pharmaceutically acceptable salt thereof, may be administered orally.

### Administration of Deferiprone and Pharmaceutical Compositions Thereof

In certain embodiments, deferiprone, or the pharmaceutically acceptable salt thereof is administered on a daily basis, for example, in one to six doses per day. In one embodiment, deferiprone or pharmaceutically acceptable salt thereof can be administered intravenously for up to three hours or less; up to two hours or less; or up to one hour or less. In certain embodiments, deferiprone or the pharmaceutically acceptable salt thereof is administered orally or intravenous. In one embodiment, the deferiprone, or the pharmaceutically acceptable salt thereof, is administered orally as part of an oral pharmaceutical composition. In another embodiment, the deferiprone, or the pharmaceutically acceptable salt thereof, is administered intravenously as part of an intravenous pharmaceutical composition.

In certain embodiments, when administered orally, a therapeutically effective amount may be 1 to 150 mg/kg, e.g., 10 to 150 mg/kg, 1 to 120 mg/kg, 1 to 50 mg/kg, 20 to 100 mg/kg, 10 to 50 mg/kg, or 50 to 100 mg/kg, of deferiprone, or an equivalent amount of the pharmaceutically acceptable salt thereof, in one or more oral doses per day, e.g., up to a maximum of 50, 100, or 150 mg/kg/day. In another embodiment, the oral pharmaceutical composition is administered at a dose of 1-50 mg/kg of deferiprone, e.g. 1-5 mg/kg, 5-10 mg/kg, 10-15 mg/kg, 15-20 mg/kg, 20-25 mg/kg, 25-30 mg/kg, 30-35 mg/kg, 35-40 mg/kg, 40-45 mg/kg, or 45-50 mg/kg of deferiprone, or an equivalent amount of the pharmaceutically acceptable salt thereof. In a further embodiment, the oral dose of deferiprone or the pharmaceutically acceptable salt thereof is 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, or 50 mg/kg. In a further embodiment, the deferiprone or the pharmaceutically acceptable salt thereof that is administered as a tablet, e.g., a tablet comprising at least 50 mg, 100 mg, 200 mg, 300 mg, 400 mg, 500 mg, 600 mg, 700 mg, 800 mg, 900 mg, or 1000 mg of deferiprone or the pharmaceutically acceptable salt thereof. In a further embodiment, the deferiprone or the pharmaceutically acceptable salt thereof that is administered as a liquid composition, e.g., a liquid composition comprising at least 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 20 mg/ml, 25 mg/ml, 30 mg/ml, 35 mg/ml, 40 mg/ml, 45 mg/ml, 50 mg/ml, or 100 mg/ml of deferiprone or the pharmaceutically acceptable salt thereof.

In one embodiment, the oral pharmaceutical composition is an immediate release, sustained release or controlled release pharmaceutical composition.

In another embodiment, the therapeutically effective amount of deferiprone may be 1 to 150 mg/kg, e.g., 10 to 150 mg/kg, 1 to 120 mg/kg, 1 to 50 mg/kg, 20 to 100 mg/kg, 10 to 50 mg/kg, or 50 to 100 mg/kg, of deferiprone, or an equivalent amount of the pharmaceutically acceptable salt thereof, in an intravenous pharmaceutical composition, in one or more oral doses per day up to a maximum of, e.g., 50, 100, or 150 mg/kg/day. In another embodiment, the intravenous pharmaceutical composition is administered at a dose of 1-50 mg/kg of deferiprone, e.g. 1-5 mg/kg, 5-10 mg/kg, 10-15 mg/kg, 15-20 mg/kg, 20-25 mg/kg, 25-30 mg/kg, 30-35 mg/kg, 35-40 mg/kg, 40-45 mg/kg, or 45-50 mg/kg of deferiprone, or an equivalent amount of the pharmaceutically acceptable salt thereof. In a further embodiment, the specific intravenous dose of deferiprone or the pharmaceutically acceptable salt thereof is 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, or 50 mg/kg. In a further embodiment, the deferiprone or the pharmaceutically acceptable salt thereof that is administered as a parenteral composition, e.g., a parenteral composition comprising at least 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 20 mg/ml, 25 mg/ml, 30 mg/ml, 35 mg/ml, 40 mg/ml, 45 mg/ml, 50 mg/ml, or 100 mg/ml of deferiprone or the pharmaceutically acceptable salt thereof.

Deferiprone, or a pharmaceutically acceptable salt thereof, is administered in any manner to achieve its intended purpose. In one embodiment, it is administered intravenously or orally. In another embodiment, deferiprone or pharmaceutically acceptable salt thereof is administered orally. In one embodiment the dosage form is a sustained release formulation made in accordance with well-known methods. Although an immediate release formulation provides adequate blood levels, a sustained release formulation will maintain a therapeutically useful level over a period of time exceeding that of an oral immediate release dose, with less fluctuation. An exemplary sustained release formulation is shown in Chart A below, as disclosed in U.S. Published Appl. 2006/0122273.

CHART A
Deferiprone Tabs with 500 mg Core

| Ingredient name | Mg/tablet |
|---|---|
| Hydroxypropyl cellulose NF | 6.0 |
| Hydroxypropyl methylcellulose USP | 1.5 |
| Polyethylene glycol 8000 NF | 4.5 |
| Titanium dioxide USP | 6.0 |
| Purified water | 132.0 |
| Sub-total | 150.0 |
| Deferiprone 500 mg core | 600.0 |
| Total (excluding water) | 618.0 |

In one embodiment, the deferiprone is administered as a monotherapy for the treatment of myocardial ischemia, an acute coronary event, e.g., a myocardial infarction, or reperfusion injury, a intramyocardial hemorrhage or the damage resulting therefrom, a cardiac edema or the damage resulting therefrom, an arrhythmia or the damage resulting therefrom, or other ischemic damage to the heart. In another embodiment, the deferiprone is coadministered with one or more second agents. The other agent or agents may be selected from the group consisting of anticoagulants, anti-platelet agents, anti-thrombins, statins, ACE inhibitors, beta-blockers, heparin, aspirin, blockers of IIb/IIa receptors hirudin, platelet-derived growth factor antagonists, coumarin, bishydroxycoumarin, warfarin, acid citrate dextrose, lepirudin, ticlopidine, clopidogrel, tirofiban, argatroban, eptifibatide, and calcitriol. In one embodiment, the deferiprone is administered as a cotherapy with an antiplatelet therapy, e.g., aspirin, clopidogrel, prasugrel, ticagrelor, ticlopidine, cilostazol, abciximab, eptifibatide, tirofiban, dipyridamole, terutroban, epoprostenol, streptokinase, a plasminogen activator, and combinations thereof.

Additional therapeutic agents useful as adjunctive therapy according to the invention include, but are not limited to, small molecules, synthetic drugs, peptides, polypeptides, proteins, nucleic acids (e.g., DNA and RNA polynucleotides including, but not limited to, antisense nucleotide sequences, triple helices, and nucleotide sequences encoding biologically active proteins, polypeptides, or peptides), antibodies, synthetic or natural inorganic molecules, mimetic agents, and synthetic or natural organic molecules. Any agent which is known to be useful, or which has been used or is currently being used for the prevention, treatment, or amelioration of myocardial infarction or reperfusion injury of a coronary artery, and/or promoting the revascularization and/or beneficial remodeling of cardiac tissue, can be used in combination with deferiprone in accordance with the invention described herein.

In certain embodiments, deferiprone is co-administered with one or more second agents. Deferiprone and the one or more second agents may be administered in a combination, separate, or sequential preparation. In one embodiment, the deferiprone is administered at the same time as the second agent. In another embodiment, the deferiprone is administered at different times as the second agent.

In one embodiment, the deferiprone is administered together with the second agent as part of a single, unitary pharmaceutical composition. In another embodiment, the deferiprone is administered together with the second agent or agents as part of separate pharmaceutical compositions.

In another embodiment, the one or more second agents may also be in a sustained release formulation either alone or together with the formulation comprising deferiprone or pharmaceutically acceptable salt thereof.

Although compositions incorporating a liquid diluent may be used for oral administration, in one embodiment a solid carrier is used, for example, a conventional solid carrier material such as starch, lactose, dextran or magnesium stearate which provides a suitable oral dosage form that is stable and does not degrade. Oral formulations may be in the form of, e.g., liquid formulations, tablets, capsules, powders.

Liquid formulations may be prepared according to well-known methods in the art, and include those disclosed in U.S. Published Appl. No. 2011/0039897, which disclosed compositions comprising deferiprone and a taste masking composition. An exemplary 100 mg/ml formulation is shown in Chart B below.

| Item | Quantity/L | |
|---|---|---|
| Deferiprone | 100.00 | g |
| Glycerin USP | 600.00 | g |
| Hydrochloric Acid NF/EP | 50 | ml |
| Hydroxyethyl cellulose NF Type H | 1.00 | g |
| Saccharin Sodium USP | 3.00 | g |
| Peppermint oil | 0.10 | g |
| Bitter blocker type flavor | 2.00 | g |
| Artificial Cherry Flavor | 2.00 | g |
| Purified water USP/EP | q.s. to 1 | L |

In another embodiment, the one or more second agents may also be in a liquid formulation either alone or together with the deferiprone or pharmaceutically acceptable salt thereof.

### Imaging

Magnetic resonance imaging (MRI) has become an important clinical tool in the non-invasive assessment of myocardial viability and function as well as detection of processes like edema and hemorrhage after AMI, thereby allowing for risk stratification in patients.

MRI has gained clinical importance in the non-invasive assessment of myocardial viability and function after AMI (64). Quantification of infarct size and extent by delayed hyperenhancement (DHE) MRI has been found to be a predictor of LV remodeling, long-term improvement, recurrent infarction, and heart failure (65). It has also been demonstrated that the presence of MVO as detected by MRI is an independent predictor of poor functional recovery after AMI while its absence is indicative of event-free survival (66, 67). A recent study by Bodi et al. (68) demonstrated that a comprehensive MRI assessment can offer prognostic value to perform risk stratification in patients with AMI beyond routine clinical markers. MRI has also been successfully employed to identify edema in AMI using elevated T2-weighted signal in both human and animal models (69-72); the area of increased signal intensity has also been correlated with area-at-risk (AAR). Shortening of T2 and T2* relaxation times has been utilized to identify hemorrhage in AMI (28, 29, 73); another report has also validated the relation between T2 and T2* and myocardial iron stores (74). T1 quantification has been recently utilized to assess methemoglobin formation in hemorrhagic infarcts (28). Furthermore, reperfused infarcts result in greater edema compared to non-reperfused infarcts. MRI parameters have been shown to be correlated with tissue water content. This can be attributed to greater inflammatory response following reperfusion.

The invention also provides a method of selecting a patient for treatment with deferiprone, or a pharmaceutically acceptable salt thereof, to treat myocardial infarction, comprising determining whether there is a hemorrhage at the place of the infarct. In one embodiment, the determining is carried out by in vivo imaging. In another embodiment, the in vivo imaging is by magnetic resonance imaging. Methods for imaging coronary arteries for the presence of hemorrhage are well known in the art. See, for example, Kim, W. Y et al., N. Encl. J. Med. 354:1863-1869 (2001); Ganame et al., European Heart Journal 30:1440-1449 (2009); and Mather et al., Heart 97:453-459 (2011).

The invention also provides a method of treating or ameliorating myocardial infarction in a patient, comprising (a) determining whether there is a hemorrhage at the place of the infarct, and (b) administering a therapeutically effective amount of deferiprone, or a pharmaceutically acceptable salt thereof, to said patient if it is determined that there is a hemorrhage at the place of the infarct. In one embodiment, the determining is carried out by in vivo imaging. In another embodiment, the in vivo imaging is by magnetic resonance imaging. Methods for imaging coronary arteries for the presence of hemorrhage are well known in the art.

### INCORPORATION BY REFERENCE

All references cited herein, including patents, patent applications, papers, text books, and the like, and the references cited therein, to the extent that they are not already, are hereby incorporated herein by reference in their entirety.

### EXAMPLES

### Example 1

### Use of Deferiprone to Effect Beneficial Remodeling in a Porcine Model of Myocardial Infarction

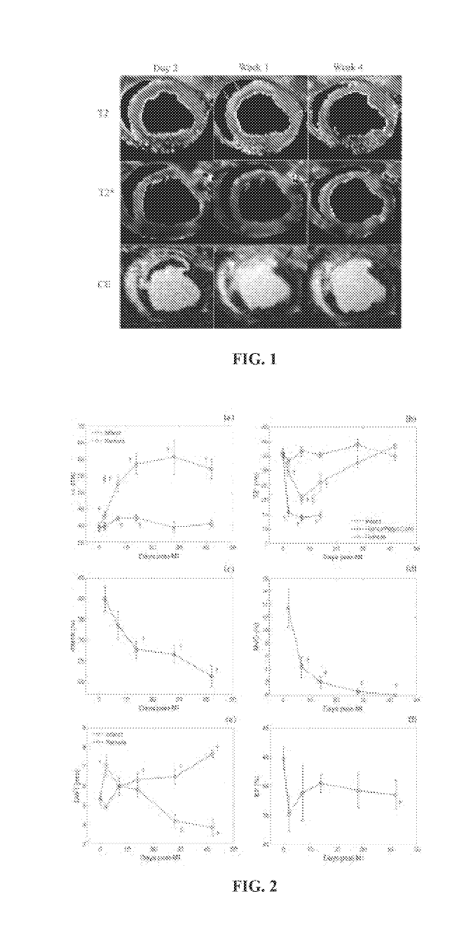

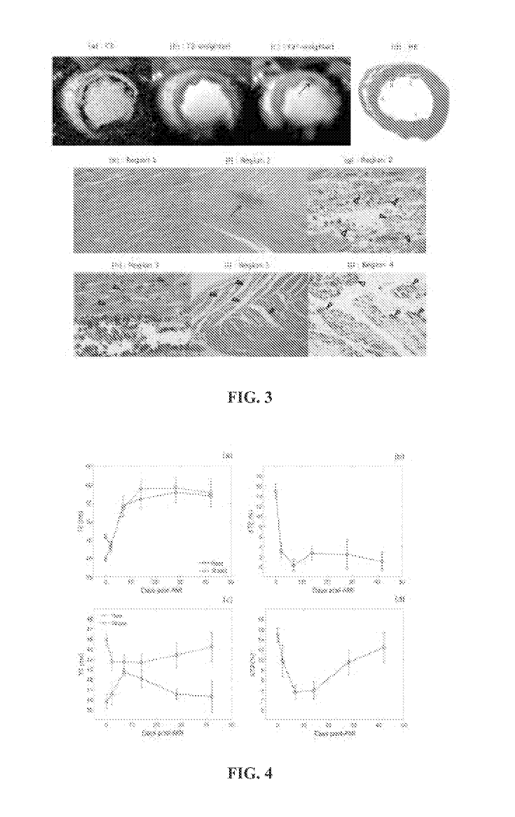

Post-infarct remodeling is a complex process with various pathophysiological changes occurring simultaneously (19) whose inter-dependence has not been completely understood in vivo. An initial study was performed to assess evolution of: 1) edema (T2); 2) hemorrhage (T2*); 3) cardiac function; 4) infarct/MVO size and 5) vasodilatory function in a porcine model of myocardial infarction. The left anterior descending artery (LAD) was occluded for 90 min followed by reperfusion; this model consistently produced anteroseptal transmural infarcts characterized by MVO. Pattern of progression of edema, hemorrhage and infarct/MVO was apparent on T2-, T2*-maps and contrast-enhanced (CE) images as shown in a representative animal (FIG. 1). Post-infarct LV remodeling was monitored by observing the cumulative time course of quantitative MRI parameters (FIG. 2). Comparison with histology revealed strong correlation with MRI markers (FIG. 3). Vasodilator function (or perfusion reserve) was evaluated with T2-based Blood-oxygen-level-dependent (BOLD) imaging using a pharmacological vasodilator (FIG. 4). The infarct zone demonstrated reduced/null stress response reflecting damaged or obstructed microvasculature; partial response may have indicated salvageable myocardium. Remote myocardium (un-infarcted, distal to infarction) exhibited impaired vasodilator function between week 1-2, which was later recovered similar to that seen by Uren et al. (75). The failure of remote vasodilatory function to regain control levels in the chronic phase of AMI potentially served as a predictor of subsequent adverse LV remodeling. The present study demonstrated that multi-parametric MRI data, acquired in a longitudinal fashion, could be employed to assess the state of myocardial tissue in vivo following AMI; the quantitative approach enabled serial, regional and cross-subject evaluation. Importantly, the evolution of MRI parameters was well correlated with histological features, similar to patterns seen in humans (24); this justified using a large animal model, e.g., porcine, for translation to the clinic. Two papers based on this work were published in MRM (32, 76).

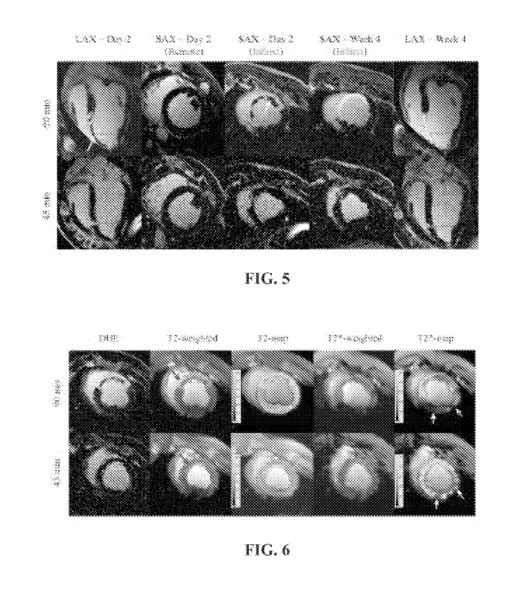

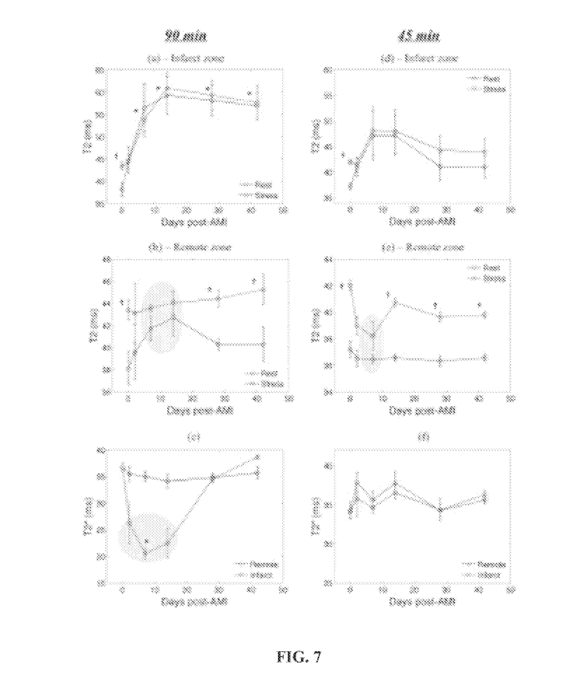

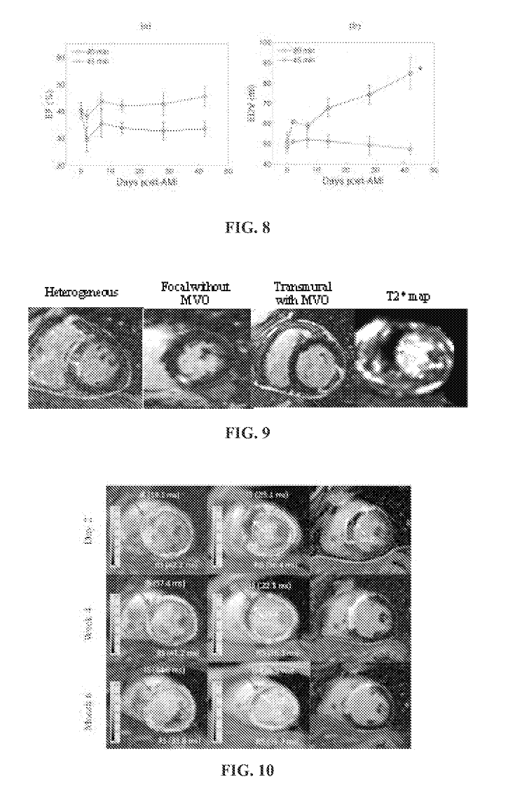

The type and extent of infarction encountered clinically (transmural, hemorrhagic, heterogeneous, with MVO) is primarily determined by the severity of the initial ischemic insult (77) or time-to-reperfusion. Understanding the evolution of remodeling mechanisms after AMI for different durations of ischemia will be key in predicting long-term functional recovery. In the current Example, two groups of pigs were studied that were subjected to different LAD occlusion durations followed by reperfusion: 90-min (N=4) and 45-min (N=3). The two groups showed distinct patterns of injury: infarcts following 90-min occlusions were transmural and hemorrhagic with MVO, while those after 45-min occlusions were small, non-hemorrhagic and heterogeneous (FIGS. 5 and 6). MRI parameters revealed faster resolution of edema (inflammation) and earlier restoration of vasodilatory function in the less severe infarcts (FIG. 7). Depressed EF and elevated EDV at week 6 (FIG. 8) in the 90-min group was suggestive of severe adverse remodeling. The study thus demonstrated that MRI evaluation could distinguish serial patterns of tissue injury based on severity of the initial ischemic insult. This may potentially allow determination of the optimal timing and duration of novel therapies in the clinical setting that are targeted to alleviate ischemic injury and prevent MVO and/or hemorrhage.

A comprehensive serial MRI examination on patients post-AMI was performed (79). The pattern of infarction was variable across patients ranging from heterogeneous to focal to transmural with MVO (FIG. 9); larger transmural infarcts sometimes developed myocardial hemorrhage as delineated by T2* images. Quantitative T2 and T2* maps were used to visualize regional and longitudinal changes in edema and hemorrhage (FIG. 10). Within the first few weeks, T2 was elevated in the infarct zone indicative of edema that was resolved by the 6-month follow-up scan while hemorrhage was resolved at weeks 2-4 (FIG. 11-top panel). Plots in FIG. 11 (bottom panel) demonstrate that quantitative T2 can detect distinct patterns of myocardial injury with and without hemorrhage. Remote zone remodeling in the hemorrhagic group may be indicative of more adverse remodeling.

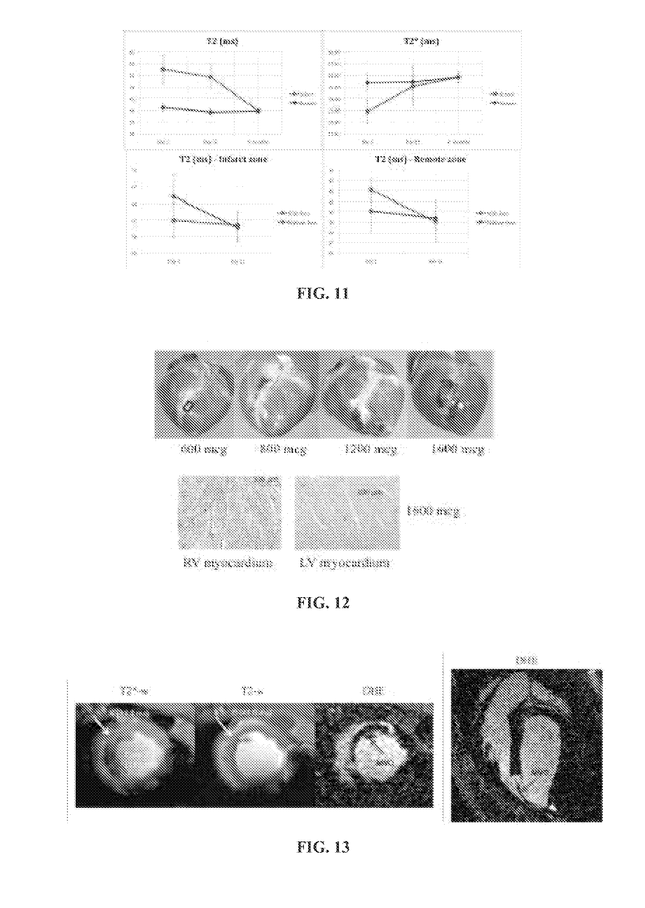

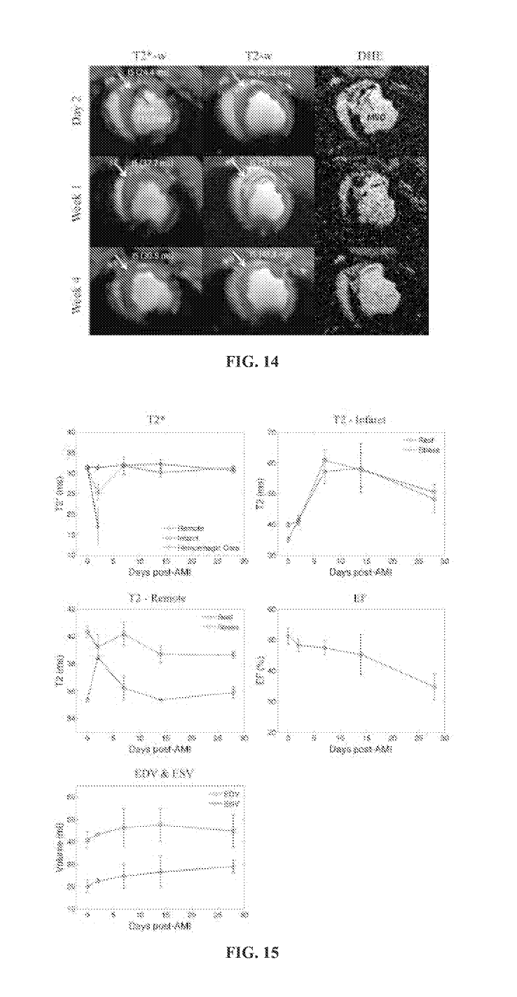

In the current example, to evaluate the contribution of hemorrhage alone in the development of adverse LV remodeling after AMI, artificially induced hemorrhage was performed in porcine hearts by direct intracoronary injection of collagenase. Collagenases are proteolytic enzymes that have been found to increase permeability of blood vessels resulting in spillage of blood into the extravascular space (81). 21 Yorkshire pigs (19-31 kg) were included in the study and 7-9 ml phosphate-buffered solution containing collagenase was injected using an over-the-wire angioplasty balloon catheter that was advanced to mid LAD after 2nd diagonal branch; balloon inflation was maintained for 8 min (ischemia). Six doses (250, 600, 800, 1200, 1600, and 3200 mcg) of collagenase were administered in equally divided groups; there was no mortality attributable to collagenase infusion. Animals were sacrificed at 24 hrs and hearts were explanted for histological analysis. Epicardial and intramyocardial hemorrhage was observed in a dose-dependent manner with none or mild focal hemorrhage up to 600 mcg, mild-moderate at 800-1600 mcg and severe at 3200 mcg (FIG. 12). After establishing this model, a pilot study was initiated to artificially induce hemorrhage in one animal subjected to a 45 min LAD occlusion, which is inherently non-hemorrhagic. Collagenase was injected immediately after balloon deflation i.e. during reperfusion at an intermediate dose of 1000 mcg. MRI examination at day 2 post-AMI revealed signal void on T2*-weighted images, indicative of hemorrhage, alongside a surprising yet interesting finding was the presence of MVO on DHE images (FIG. 13). This was unlike the untreated 45 min infarcts studied. Currently, the causality between hemorrhage and MVO is unknown. The blood spilt in the interstitium might have compressed the microvasculature that was already vulnerable due to the initial ischemic insult; in other words, hemorrhage may have created the MVO.

Excess tissue iron accumulation can be lethal as free iron is toxic in nature. It has been speculated that iron chelation may be beneficial in acute coronary syndromes and that early treatment can limit ischemia-reperfusion injury and also reduce infarct size (83, 84). However, the role of iron chelation in hemorrhagic infarction has not yet been explored. In the present example, the cardioprotective properties of the iron chelating agent deferiprone (DFP) were explored in a porcine model of myocardial infarction. Deferiprone offered several advantages in that it is a very small molecule, both hydrophilic and lipophilic and highly membrane permeable; it can readily enter mitochondria and may access intracellular labile iron. In a pilot study, DFP was administered (orally) to pigs (N=2) a few hours before a 90 min LAD occlusion (pre-loading), and treatment was continued with a daily dose of 100 mg/kg (85). Animals were then monitored with a comprehensive MRI exam from day 2 to week 4. Key highlights of the study were: 1) Hemorrhage (from T2* image) was observed only on day 2 and by week 1 it had completely resolved. This was in contrast to the untreated group (FIG. 2) where resolution of hemorrhage was delayed to week 4; 2) Inflammation or edema was substantially reduced by week 4 with T2 values approaching control levels. In the untreated group, edema persisted even up to week 6; 3) Vasodilatory dysfunction was observed only at day 2 with recovery from then onwards, whereas the untreated group demonstrated dysfunction up to week 2; 4) EF was gradually depressed by week 4 probably due to the severity of the initial insult; 5) Ventricular volume was relatively unchanged suggesting little or no preload stress, unlike the untreated group. FIG. 14 demonstrates representative images and FIG. 15 shows the cumulative time course of the MRI measurements. No side effects of DFP were observed in the animals throughout the 4 weeks of observation. The result shown in FIG. 14 compared to FIG. 1 show the neutralizing capacity of DFP. These findings suggested that DFP was able to penetrate the infarct zone despite the MVO and was also effective in neutralizing hemorrhagic byproducts and reducing edema—representing a beneficial remodeling process.

### Example 2

### Comparison of Deferoxamine, Deferasirox and Deferiprone in a Porcine Model of Myocardial Infarction

Excess tissue iron deposition/accumulation can be lethal as free iron is toxic in nature. Iron chelators have been shown to be a lifeline for patients with iron overload syndromes and the benefits have been well demonstrated in both clinical and preclinical environments (86, 87). This presents a distinctly different situation than is seen in subjects who do not have iron overload, but develop myocardial ischemia and/or a myocardial infarction. Since the former represents years of exposure to very high levels of cardiac iron, several fold greater than that present in patients with an acute MI, it is invalid to extrapolate the findings from patients with iron overload to others, both because the time of iron exposure and the concentration of labile iron are orders of magnitude different. Furthermore, the use of chelators in iron overload is not for an acute event, as is the case for this application, but for long term prevention of the consequences of iron overload. Thus, although it has also been speculated that iron chelation may be beneficial in acute coronary syndromes and that early treatment can limit ischemia-reperfusion injury and also reduce infarct size (88, 89), there are no data to support such speculation. The benefit anticipated in these documents is on the basis of reperfusion injury. However, as noted above, the magnitude of excess iron in these circumstances does not begin to approach the levels seen in transfusional iron overload, and it is our view that iron chelation of ROS-mediated activity resulting from reperfusion injury, would be insufficient to effect a meaningful correction of the induced pathology with an MI, in the absence of other changes to the myocardial tissue. For example, treatment with the iron chelating agent deferoxamine (DFO) was shown to reduce reactive oxygen species (ROS) (F2-isoprostane), but have no significant effect on infarct size, creatinine kinase or Troponin-1 in patients with myocardial infarction (Chan et al., Circ. Cardiovasc Interv. 5:270-278 (2012)). We have executed a study to establish if an iron chelator, specifically deferiprone, could have a favorable effect, and if it did so, what changes would be evident in the myocardium that would account for those beneficial changes. As revealed in Example 1, deferiprone had a strong effect and the effects were attributable to new vascularization and remodeling, beyond simple prevention of ROS-mediated toxicity pursuant to reperfusion injury.

A next step would be to define whether this is a benefit to be expected from all currently available iron chelators, or just from deferiprone. Thus, the study of all 3 iron chelating agents in our porcine model of myocardial infarction provides the ability to compare their cardioprotective properties.

A porcine model of myocardial infarction is used in this study. The study utilizes female Yorkshire pigs (20-25 kg, University of Guelph, Guelph, Ontario, Canada) and procedures are conducted in accordance with protocols approved by the Animal Care Committee of Sunnybrook Heath Sciences Centre. Animals are brought under sedation with an anesthetic cocktail comprising atropine (0.05 mg/kg) and ketamine (30 mg/kg). Animals are then intubated and respiration is controlled (20-25 breaths/min) with a mechanical ventilator along with inhalation of isoflurane (1-5%) for maintaining anesthesia. Myocardial infarction is achieved by complete coronary occlusion distal to the second diagonal branch of the left anterior descending artery (LAD) for 90 minutes via inflation of a percutaneous balloon dilation catheter (Sprinter Legend Balloon Catheter, Medtronic, Minneapolis, Minn.), that is followed by reperfusion. Upon balloon removal, restoration of blood flow through the artery is verified. X-ray fluoroscopy (OEC 9800, GE Healthcare, Milwaukee, Wis.) of iodinated contrast distribution is employed for guiding balloon placement/inflation and noting coronary blood flow patterns. An intravenous (IV) line is created via the ear vein for the administration of maintenance fluids. All animals are recovered for MRI scanning following the interventional procedure.

Three widely accepted chelators (chelation group) are used in the study whose dose regimens are those relevant to safe and effective doses in humans to enable a meaningful utilization of the data: (1) deferoxamine (DFO) is injected at a dose of 10-60 mg/kg/day, and 5 days/week; (2) deferasirox (DFX) is an oral chelator with a dose of 20-40 mg/kg, daily; and (3) deferiprone (DFP) is an oral chelator with a dose of 50-100 mg/kg daily.

In each chelation group, animals are subjected to myocardial infarction with the procedure described above. Animals are subjected to iron chelation starting a few hours before the infarction procedure (preload), at the time of reperfusion by injecting directly into the occluded coronary artery and continued daily treatment until sacrifice. Animals are monitored throughout infarct healing (Day 2 to week 4) by comprehensive MRI examinations to track edema, hemorrhage, vasodilatory function, infarct and microvascular obstruction size along with cardiac function; these quantitative markers are used for evaluating the effects of iron chelation on tissue remodeling following myocardial infarction. The value of quantitative MRI for monitoring adverse remodeling mechanisms in a porcine model of myocardial infarction has been previously demonstrated (99). Animals are sacrificed either at week 1 or week 4 for histological analysis on stained tissue sections in order to obtain ground truth regarding underlying pathophysiological processes. In addition, blood samples are drawn at various times post-infarction for evaluating oxidative status and inflammatory response. The outcome regarding effective and beneficial effect for deferiprone in terms of recovery, including favorable remodeling compared to the effects of DFO and DFX will be determined.

### Example 3

### Effect of Deferiprone in Hemorrhagic Myocardial Infarction

A quantitative study was done to investigate the effect of the deferiprone (DFP) on the hemorrhagic byproducts in tissue in a porcine model of myocardial infarction, and to monitor remodeling by cardiovascular magnetic resonance (CMR).

The study involved two groups of animals that were subjected to a 90 min balloon occlusion of the left anterior descending artery (LAD) followed by reperfusion—untreated (N=2) and DFP treated (N=2). DFP was administered (orally) a few hours (about 1-2 hours) before the procedure (pre-loading) and treatment was continued with a daily dose of 100 mg/kg. Imaging was performed on a 3T MRI scanner (MR 750, GE Healthcare) pre-AMI (healthy), day 2, week 1, week 2 and week 4 post-AMI. Edema was evaluated by T2 quantification using a T2-prepared spiral sequence and hemorrhage was identified by T2* using a multi-echo gradient-echo acquisition. Infarct assessment was performed by delayed hyperenhancement (DHE) using an IR-GRE sequence.

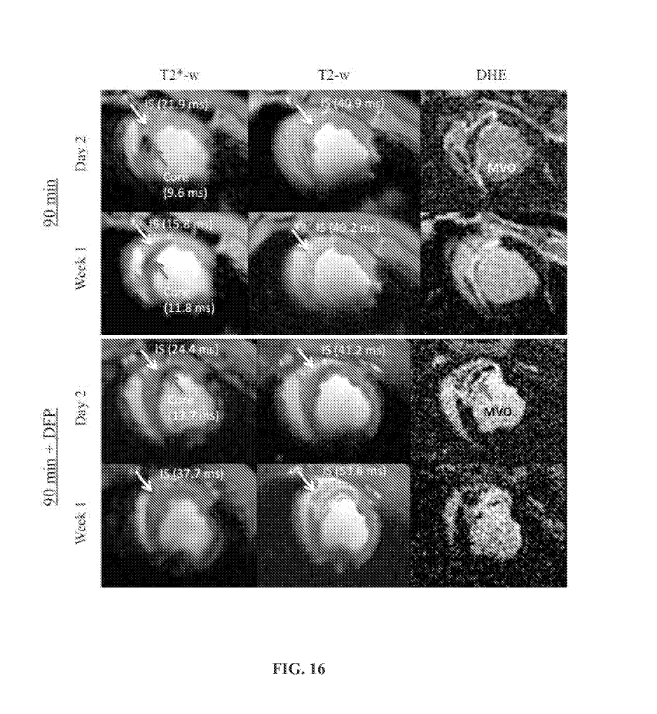

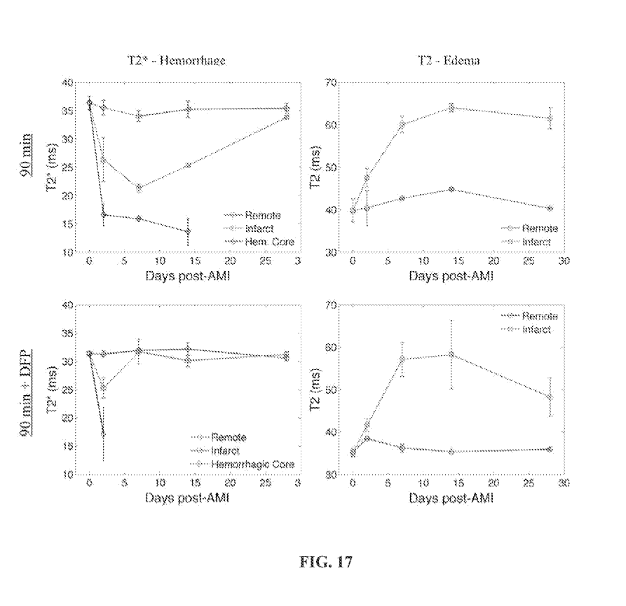

FIG. 16 shows representative images from the two groups and FIG. 17 shows the cumulative time course of the CMR measurements.

In the DFP treated group, hemorrhage (as indicated by T2* image) was observed only on day 2 and by week 1 it had completely resolved. In the untreated group, resolution of hemorrhage was delayed to week 4. In both groups, microvascular obstruction (MVO) was seen on day 2 and was partially resolved by week 1.

With DFP treatment, inflammation and edema was substantially reduced by week 4 with T2 values approaching control levels. In the untreated group, edema persisted up to week 4. Ejection fraction (EF) was depressed by week 4 in both groups. However, end-diastolic and end-systolic volumes were relatively unchanged in the DFP treated group while they increased significantly in the untreated group.

DFP was able to penetrate the infarct zone and was also effective in neutralizing hemorrhagic byproducts. Elimination of hemorrhage resulted in faster resolution of edema and normal ventricular volumes, representing a beneficial remodeling process.

All patents, patent applications and publications cited herein are fully incorporated by reference herein.
1. Statistics Canada, Leading Causes of deaths in Canada, 2007, CANSIM Table 102-0561, Published November 2010.
2. Van de Werf F, Ardissino D, Betriu A, Cokkinos D V, Falk E, Fox K A, Julian D, Lengyel M, Neumann F J, Ruzyllo W, Thygesen C, Underwood S R, Vahanian A, Verheugt F W, Wijns W. Management of acute myocardial infarction in patients presenting with ST-segment elevation. The Task Force on the Management of Acute Myocardial Infarction of the European Society of Cardiology. Eur Heart J 2003; 24(1):28-66.
3. Van de Werf F, Bax J, Betriu A, Blomstrom-Lundqvist C, Crea F, Falk V, Filippatos G, Fox K, Huber K, Kastrati A, Rosengren A, Steg P G, Tubaro M, Verheugt F, Weidinger F, Weis M. Management of acute myocardial infarction in patients presenting with persistent ST-segment elevation: the Task Force on the Management of ST-Segment Elevation Acute Myocardial Infarction of the European Society of Cardiology. Eur Heart J 2008; 29(23):2909-2945.
4. Lambert L, Brown K, Segal E, Brophy J, Rodes-Cabau J, Bogaty P. Association between timeliness of reperfusion therapy and clinical outcomes in ST-elevation myocardial infarction. JAMA 2010; 303(21):2148-2155.
5. Rozenman Y, Gotsman M S. The earliest diagnosis of acute myocardial infarction. Annu Rev Med 1994; 45:31-44.
6. Reimer K A, Jennings R B. The “wavefront phenomenon” of myocardial ischemic cell death. II. Transmural progression of necrosis within the framework of ischemic bed size (myocardium at risk) and collateral flow. Lab Invest 1979; 40(6):633-644.
7. De Luca G, Suryapranata H, Zijlstra F, van't H of A W, Hoorntje J C, Gosselink A T, Dambrink J H, de Boer M J. Symptom-onset-to-balloon time and mortality in patients with acute myocardial infarction treated by primary angioplasty. J Am CollCardiol 2003; 42(6):991-997.
8. Eitel I, Desch S, Fuernau G, Hildebrand L, Gutberlet M, Schuler G, Thiele H. Prognostic significance and determinants of myocardial salvage assessed by cardiovascular magnetic resonance in acute reperfused myocardial infarction. Am Coll Cardiol; 55(22):2470-2479.
9. Hudson M P, Armstrong P W, O'Neil W W, Stebbins A L, Weaver W D, Widimsky P, Aylward P E, Ruzyllo W, Holmes D, Mahaffey K W, Granger C B. Mortality Implications of Primary Percutaneous Coronary Intervention Treatment Delays Insights From the Assessment of Pexelizumab in Acute Myocardial Infarction Trial. CircCardiovascQual Outcomes 2011.
10. Yellon D M, Hausenloy D J. Myocardial reperfusion injury. N Engl J Med 2007; 357(11):1121-1135.
11. Jennings R B, Schaper J, Hill M L, Steenbergen C, Jr., Reimer K A. Effect of reperfusion late in the phase of reversible ischemic injury. Changes in cell volume, electrolytes, metabolites, and ultrastructure. Circ Res 1985; 56(2):262-278.
12. Wu K C, Zerhouni E A, Judd R M, Lugo-Olivieri C H, Barouch L A, Schulman S P, Blumenthal R S, Lima J A. Prognostic significance of microvascular obstruction by magnetic resonance imaging in patients with acute myocardial infarction. Circulation 1998; 97(8):765-772.
13. Fishbein M C, J Y R, Lando U, Kanmatsuse K, Mercier J C, Ganz W. The relationship of vascular injury and myocardial hemorrhage to necrosis after reperfusion. Circulation 1980; 62(6):1274-1279.
14. Ganame J, Messalli G, Dymarkowski S, Rademakers F E, Desmet W, Van de Werf F, Bogaert J. Impact of myocardial haemorrhage on left ventricular function and remodelling in patients with reperfused acute myocardial infarction. Eur Heart J 2009; 30(12):1440-1449.
15. Ye Y, Perez-Polo J R, Birnbaum Y. Protecting against ischemia-reperfusion injury: antiplatelet drugs, statins, and their potential interactions Ann N Y Acad Sci 2010; 1207:76-82.
16. Landmesser U, Wollert K C, Drexler H. Potential novel pharmacological therapies for myocardial remodelling. Cardiovasc Res 2009; 81(3):519-527.
17. Bolognese L, Carrabba N, Parodi G, Santoro G M, Buonamici P, Cerisano G, Antoniucci D. Impact of microvascular dysfunction on left ventricular remodeling and long-term clinical outcome after primary coronary angioplasty for acute myocardial infarction. Circulation 2004; 109(9):1121-1126.
18. Bolognese L, Neskovic A N, Parodi G, Cerisano G, Buonamici P, Santoro G M, Antoniucci D. Left ventricular remodeling after primary coronary angioplasty: patterns of left ventricular dilation and long-term prognostic implications. Circulation 2002; 106(18):2351-2357.
19. Jugdutt B I. Ventricular remodeling after infarction and the extracellular collagen matrix: when is enough enough? Circulation 2003; 108(11):1395-1403.
20. Bodi V, Sanchis J, Nunez J, Mainar L, Minana G, Benet I, Solano C, Chorro F J, Llacer A. Uncontrolled immune response in acute myocardial infarction: unraveling the thread. Am Heart J 2008; 156(6):1065-1073.
21. Garcia S, Henry T D, Wang Y L, Chavez I J, Pedersen W R, Lesser J R, Shroff G R, Moore L, Traverse J H. Long-term follow-up of patients undergoing postconditioning during ST-elevation myocardial infarction. J CardiovascTransl Res; 4(1):92-98.
22. Botker H E, Kharbanda R, Schmidt M R, Bottcher M, Kaltoft A K, Terkelsen C J, Munk K, Andersen N H, Hansen T M, Trautner S, Lassen J F, Christiansen E H, Krusell L R, Kristensen S D, Thuesen L, Nielsen S S, Rehling M, Sorensen H T, Redington A N, Nielsen T T. Remote ischaemic conditioning before hospital admission, as a complement to angioplasty, and effect on myocardial salvage in patients with acute myocardial infarction: a randomised trial. Lancet 2010; 375(9716):727-734.
23. Freixa X, Bellera N, Ortiz-Perez J T, Jimenez M, Pare C, Bosch X, De Caralt T M, Betriu A, Masotti M. Ischaemicpostconditioning revisited: lack of effects on infarct size following primary percutaneous coronary intervention. Eur Heart J.
24. Fishbein M C, Maclean D, Maroko P R. The histopathologic evolution of myocardial infarction. Chest 1978; 73 (6): 843-849.
25. Garcia-Dorado D, Theroux P, Solares J, Alonso J, Fernandez-Aviles F, Elizaga J, Soriano J, Botas J, Munoz R. Determinants of hemorrhagic infarcts. Histologic observations from experiments involving coronary occlusion, coronary reperfusion, and reocclusion. Am J Pathol 1990; 137(2):301-311.
26. Higginson L A, Beanlands D S, Nair R C, Temple V, Sheldrick K. The time course and characterization of myocardial hemorrhage after coronary reperfusion in the anesthetized dog. Circulation 1983; 67(5):1024-1031.
27. Bekkers S C, Smulders M W, Passos V L, Leiner T, Waltenberger J, Gorgels A P, Schalla S. Clinical implications of microvascular obstruction and intramyocardialhaemorrhage in acute myocardial infarction using cardiovascular magnetic resonance imaging. EurRadiol 2010; 20(11):2572-2578.
28. Foltz W D, Yang Y, Graham J J, Detsky J S, Wright G A, Dick A J. MRI relaxation fluctuations in acute reperfused hemorrhagic infarction. MagnReson Med 2006; 56(6):1311-1319.
29. O'Regan D P, Ahmed R, Karunanithy N, Neuwirth C, Tan Y, Durighel G, Hajnal J V, Nadra I, Corbett S J, Cook S A. Reperfusion hemorrhage following acute myocardial infarction: assessment with T2* mapping and effect on measuring the area at risk. Radiology 2009; 250(3):916-922.
30. Mather A N, Fairbairn T A, Ball S G, Greenwood J P, Plein S. Reperfusion haemorrhage as determined by cardiovascular MRI is a predictor of adverse left ventricular remodelling and markers of late arrhythmic risk. Heart 2011; 97(6):453-459.
31. Bekkers S C, Backes W H, Kim R J, Snoep G, Gorgels A P, Passos V L, Waltenberger J, Crijns H J, Schalla S. Detection and characteristics of microvascular obstruction in reperfused acute myocardial infarction using an optimized protocol for contrast-enhanced cardiovascular magnetic resonance imaging. EurRadiol 2009.
32. Ghugre N R, Ramanan V, Pop M, Yang Y, Barry J, Qiang B, Connelly K, Dick A J, Wright G A. Quantitative tracking of edema, hemorrhage and microvascular obstruction in sub-acute myocardial infarction in a porcine model by MRI. MagnReson Med 2011 (February 17, Epub ahead of print).
33. Cannan C, Eitel I, Hare J, Kumar A, Friedrich M. Hemorrhage in the myocardium following infarction. JACC Cardiovasc Imaging 2010; 3(6):665-668.
34. Roberts C S, Schoen F J, Kloner R A. Effect of coronary reperfusion on myocardial hemorrhage and infarct healing. Am J Cardiol 1983; 52(5):610-614.
35. Reimer K A, Jennings R B. The changing anatomic reference base of evolving myocardial infarction. Underestimation of myocardial collateral blood flow and overestimation of experimental anatomic infarct size due to tissue edema, hemorrhage and acute inflammation. Circulation 1979; 60(4):866-876.
36. Pislaru S V, Barrios L, Stassen T, Jun L, Pislaru C, Van de Werf F. Infarct size, myocardial hemorrhage, and recovery of function after mechanical versus pharmacological reperfusion: effects of lytic state and occlusion time. Circulation 1997; 96(2):659-666.
37. Reffelmann T, Kloner R A. The “no-reflow” phenomenon: basic science and clinical correlates. Heart 2002; 87(2):162-168.
38. Mather A N, Fairbairn T A, Ball S G, Greenwood J P, Plein S. Reperfusion haemorrhage as determined by cardiovascular MRI is a predictor of adverse left ventricular remodelling and markers of late arrhythmic risk. Heart; 97(6):453-459.
39. Bradley W G, Jr. MR appearance of hemorrhage in the brain. Radiology 1993; 189(1):15-26.
40. Okauchi M, Hua Y, Keep R F, Morgenstern L B, Xi G. Effects of deferoxamine on intracerebral hemorrhage-induced brain injury in aged rats. Stroke 2009; 40(5):1858-1863.
41. Huang F P, Xi G, Keep R F, Hua Y, Nemoianu A, Hoff J T. Brain edema after experimental intracerebral hemorrhage: role of hemoglobin degradation products. J Neurosurg 2002; 96(2):287-293.
42. Leung G, Moody A R. MR imaging depicts oxidative stress induced by methemoglobin. Radiology 2010; 257(2):470-476.
43. Kalinowski D S, Richardson D R. The evolution of iron chelators for the treatment of iron overload disease and cancer. Pharmacol Rev 2005; 57(4):547-583.
44. Hershko C, Link G, Konijn A M, Cabantchik Z I. Objectives and mechanism of iron chelation therapy. Ann N Y Acad Sci 2005; 1054:124-135.
45. Szuber N, Buss J L, Soe-Lin S, Felfly H, Trudel M, Ponka P. Alternative treatment paradigm for thalassemia using iron chelators. ExpHematol 2008; 36(7):773-785.
46. Shalev O, Repka T, Goldfarb A, Grinberg L, Abrahamov A, Olivieri N F, Rachmilewitz E A, Hebbel R P. Deferiprone (L1) chelates pathologic iron deposits from membranes of intact thalassemic and sickle red blood cells both in vitro and in vivo. Blood 1995; 86(5):2008-2013.
47. Kwong R Y, Pfeffer M A. Infarct haemorrhage detected by cardiac magnetic resonance imaging: are we seeing the latest culprit in adverse left ventricular remodelling? Eur Heart J 2009; 30(12):1431-1433.
48. Steffens S, Montecucco F, Mach F. The inflammatory response as a target to reduce myocardial ischaemia and reperfusion injury. ThrombHaemost 2009; 102(2):240-247.
49. Nian M, Lee P, Khaper N, Liu P. Inflammatory cytokines and postmyocardial infarction remodeling. Circ Res 2004; 94(12):1543-1553.
50. Frangogiannis N G. The immune system and cardiac repair. Pharmacol Res 2008; 58(2):88-111.
51. Frangogiannis N G, Smith C W, Entman M L. The inflammatory response in myocardial infarction. Cardiovasc Res 2002; 53(1):31-47.
52. Ono K, Matsumori A, Shioi T, Furukawa Y, Sasayama S. Cytokine gene expression after myocardial infarction in rat hearts: possible implication in left ventricular remodeling. Circulation 1998; 98(2):149-156.
53. Finkel M S, Oddis C V, Jacob T D, Watkins S C, Hattler B G, Simmons R L. Negative inotropic effects of cytokines on the heart mediated by nitric oxide. Science 1992; 257(5068):387-389.
54. Yokoyama T, Vaca L, Rossen R D, Durante W, Hazarika P, Mann D L. Cellular basis for the negative inotropic effects of tumor necrosis factor-alpha in the adult mammalian heart. J Clin Invest 1993; 92(5):2303-2312.
55. Siwik D A, Chang D L, Colucci W S. Interleukin-1beta and tumor necrosis factor-alpha decrease collagen synthesis and increase matrix metalloproteinase activity in cardiac fibroblasts in vitro. Circ Res 2000; 86(12):1259-1265.
56. Maekawa N, Wada H, Kanda T, Niwa T, Yamada Y, Saito K, Fujiwara H, Sekikawa K, Seishima M. Improved myocardial ischemia/reperfusion injury in mice lacking tumor necrosis factor-alpha. J Am CollCardiol 2002; 39(7):1229-1235.
57. Mosmann T R. Properties and functions of interleukin-10. AdvImmunol 1994; 56:1-26.
58. Moore K W, O'Garra A, de Waal Malefyt R, Vieira P, Mosmann T R. Interleukin-10. Annu Rev Immunol 1993; 11:165-190.
59. Lacraz S, Nicod L P, Chicheportiche R, Welgus H G, Dayer J M. IL-10 inhibits metalloproteinase and stimulates TIMP-1 production in human mononuclear phagocytes. J Clin Invest 1995; 96(5):2304-2310.
60. Libby P, Maroko P R, Bloor C M, Sobel B E, Braunwald E. Reduction of experimental myocardial infarct size by corticosteroid administration. J Clin Invest 1973; 52(3):599-607.
61. Jugdutt B I, Basualdo C A. Myocardial infarct expansion during indomethacin or ibuprofen therapy for symptomatic post infarction pericarditis. Influence of other pharmacologic agents during early remodelling. Can J Cardiol 1989; 5(4):211-221.
62. Roberts R, DeMello V, Sobel B E. Deleterious effects of methylprednisolone in patients with myocardial infarction. Circulation 1976; 53(3 Suppl):1204-206.
63. Cai B, Deitch E A, Ulloa L. Novel insights for systemic inflammation in sepsis and hemorrhage. Mediators Inflamm 2010; 2010:642462.
64. West A M, Kramer C M. Cardiovascular Magnetic Resonance Imaging of Myocardial Infarction, Viability, and Cardiomyopathies. CurrProblCardiol 2010; 35(4):176-220.
65. Choi K M, Kim R J, Gubernikoff G, Vargas J D, Parker M, Judd R M. Transmural extent of acute myocardial infarction predicts long-term improvement in contractile function. Circulation 2001; 104 (10):1101-1107.
66. Rogers W J, Jr., Kramer C M, Geskin G, Hu Y L, Theobald T M, Vido D A, Petruolo S, Reichek N. Early contrast-enhanced MRI predicts late functional recovery after reperfused myocardial infarction. Circulation 1999; 99(6):744-750.
67. Choi C J, Haji-Momenian S, Dimaria J M, Epstein F H, Bove C M, Rogers W J, Kramer C M. Infarct involution and improved function during healing of acute myocardial infarction: the role of microvascular obstruction. J CardiovascMagnReson 2004; 6(4):917-925.
68. Bodi V, Sanchis J, Nunez J, Mainar L, Lopez-Lereu M P, Monmeneu J V, Rumiz E, Chaustre F, Trapero I, Husser O, Forteza M J, Chorro F J, Llacer A. Prognostic value of a comprehensive cardiac magnetic resonance assessment soon after a first ST-segment elevation myocardial infarction. JACC Cardiovasc Imaging 2009; 2(7):835-842.
69. Aletras A H, Tilak G S, Natanzon A, Hsu L Y, Gonzalez F M, Hoyt R F, Jr., Arai A E. Retrospective determination of the area at risk for reperfused acute myocardial infarction with T2-weighted cardiac magnetic resonance imaging: histopathological and displacement encoding with stimulated echoes (DENSE) functional validations. Circulation 2006; 113(15):1865-1870.
70. Friedrich M G, Abdel-Aty H, Taylor A, Schulz-Menger J, Messroghli D, Dietz R. The salvaged area at risk in reperfused acute myocardial infarction as visualized by cardiovascular magnetic resonance. J Am CollCardiol 2008; 51(16):1581-1587.
71. Abdel-Aty H, Simonetti O, Friedrich M G. T2-weighted cardiovascular magnetic resonance imaging. J MagnReson Imaging 2007; 26(3):452-459.
72. Giri S, Chung Y C, Merchant A, Mihai G, Rajagopalan S, Raman S V, Simonetti O P. T2 quantification for improved detection of myocardial edema. J CardiovascMagnReson 2009; 11(1):56.
73. Lotan C S, Miller S K, Bouchard A, Cranney G B, Reeves R C, Bishop S P, Elgavish G A, Pohost G M. Detection of intramyocardial hemorrhage using high-field proton (1H) nuclear magnetic resonance imaging. CathetCardiovascDiagn 1990; 20(3):205-211.
74. Ghugre N R, Enriquez C M, Gonzalez I, Nelson M D, Jr., Coates T D, Wood J C. MRI detects myocardial iron in the human heart. MagnReson Med 2006; 56(3):681-686.
75. Uren N G, Crake T, Lefroy D C, de Silva R, Davies G J, Maseri A. Reduced coronary vasodilator function in infarcted and normal myocardium after myocardial infarction. N Engl J Med 1994; 331(4):222-227.
76. Ghugre N R, Ramanan V, Pop M, Yang Y, Barry J, Qiang B, Connelly K, Dick A J, Wright G A. Myocardial BOLD imaging at 3T using quantitative T2: Application in a myocardial infarct model. MagnReson Med 2011 (May 31, Epub ahead of print).
77. Tarantini G, Cacciavillani L, Corbetti F, Ramondo A, Marra M P, Bacchiega E, Napodano M, Bilato C, Razzolini R, Iliceto S. Duration of ischemia is a major determinant of transmurality and severe microvascular obstruction after primary angioplasty: a study performed with contrast-enhanced magnetic resonance. J Am CollCardiol 2005; 46(7):1229-1235.
78. Ghugre N, Barry J, Qiang B, Graham J, Connelly K, Dick A, Wright G. Longitudinal trends of remodeling mechanisms after acute myocardial infarction based on severity of ischemic insult: A quantitative MRI study. Journal of Cardiovascular Magnetic Resonance 2011; 13(Suppl 1):057.
79. Zia M, Ghugre N, Paul G, Stainsby J, Ramanan V, Connelly K, Wright G, Dick A. Characterizing myocardial edema and hemorrhage using T2, T2*, and diastolic wall thickness post acute myocardial infarction. Journal of Cardiovascular Magnetic Resonance 2010; 12(Suppl 1):P179.
80. Zia M, Ghugre N, Connelly K, Wright G, Dick A. Thrombus aspiration during primary percutaneous coronary intervention leads to reduced myocardial edema and microvascular obstruction in infarct segment post acute myocardial infarction. Journal of Cardiovascular Magnetic Resonance 2011; 13(Suppl 1):P133.
81. Robert A M, Godeau G. Action of proteolytic and glycolytic enzymes on the permeability of the blood-brain barrier. Biomedicine 1974; 21(1):36-39.
82. Rosenberg G A, Mun-Bryce S, Wesley M, Kornfeld M. Collagenase-induced intracerebral hemorrhage in rats. Stroke 1990; 21(5):801-807.
83. Dendorfer A, Heidbreder M, Hellwig-Burgel T, Johren O, Qadri F, Dominiak P. Deferoxamine induces prolonged cardiac preconditioning via accumulation of oxygen radicals. Free RadicBiol Med 2005; 38(1):117-124.
84. Reddy B R, Kloner R A, Przyklenk K. Early treatment with deferoxamine limits myocardial ischemic/reperfusion injury. Free RadicBiol Med 1989; 7(1):45-52.
85. Wood J C, Aguilar M, Otto-Duessel M, Nick H, Nelson M D, Moats R. Influence of iron chelation on R1 and R2 calibration curves in gerbil liver and heart. MagnReson Med 2008; 60(1):82-89.
86. Hershko C, Link G, Konijn A M, Cabantchik Z I. Objectives and mechanism of iron chelation therapy. Ann N Y Acad Sci 2005; 1054:124-135.
87. Wood J C, Otto-Duessel M, Gonzalez I, Aguilar M I, Shimada H, Nick H, Nelson M, Moats R. Deferasirox and deferiprone remove cardiac iron in the iron-overloaded gerbil. Transl Res 2006; 148(5):272-280.
88. Dendorfer A, Heidbreder M, Hellwig-Burgel T, Johren O, Qadri F, Dominiak P. Deferoxamine induces prolonged cardiac preconditioning via accumulation of oxygen radicals. Free Radic Biol Med 2005; 38(1):117-124.
89. Reddy B R, Kloner R A, Przyklenk K. Early treatment with deferoxamine limits myocardial ischemic/reperfusion injury. Free Radic Biol Med 1989; 7(1):45-52.
90. Wood J C, Aguilar M, Otto-Duessel M, Nick H, Nelson M D, Moats R. Influence of iron chelation on R1 and R2 calibration curves in gerbil liver and heart. Magn Reson Med 2008; 60(1):82-89.
91. Ghugre N R, Ramanan V, Pop M, Yang Y, Barry J, Qiang B, Connelly K A, Dick A J, Wright G A. Quantitative tracking of edema, hemorrhage, and microvascular obstruction in subacute myocardial infarction in a porcine model by MRI. Magn Reson Med 2011; 66(4):1129-1141.
92. Paraskevaidis I A, Iliodromitis E K, Vlahakos D, Tsiapras D P, Nikolaidis A, Marathias A, Michalis A, Kremastinos D T. Deferoxamine infusion during coronary artery bypass grafting ameliorates lipid peroxidation and protects the myocardium against reperfusion injury: immediate and long-term significance. Eur Heart J. 2005 February; 26(3):263-70. Epub 2004 Nov. 30. PubMed PMID: 15618054.
93. Menasch P, Antebi H, Alcindor L G, Teiger E, Perez G, Giudicelli Y, Nordmann R, Piwnica A. Iron chelation by deferoxamine inhibits lipid peroxidation during cardiopulmonary bypass in humans. Circulation. 1990 November; 82(5 Suppl):IV390-6. PubMed PMID: 2225430.
94. Ferreira R, Burgos M, Milei J, Llesuy S, Molteni L, Hourquebie H, Boveris A. Effect of supplementing cardioplegic solution with deferoxamine on reperfused human myocardium. J Thorac Cardiovasc Surg. 1990 November; 100(5):708-14. PubMed PMID: 2232833.
95. Reddy B R, Wynne J, Kloner R A, Przyklenk K. Pretreatment with the iron chelator desferrioxamine fails to provide sustained protection against myocardial ischaemia-reperfusion injury. Cardiovasc Res. 1991 September; 25(9):711-8. PubMed PMID: 1799904.
96. Lesnefsky E J, Hedlund B E, Hallaway P E, Horwitz L D. High-dose iron-chelator therapy during reperfusion with deferoxamine-hydroxyethyl starch conjugate fails to reduce canine infarct size. J Cardiovasc Pharmacol. 1990 October; 16(4):523-8. PubMed PMID: 1706792.
97. Reddy B R, Kloner R A, Przyklenk K. Early treatment with deferoxamine limits myocardial ischemic/reperfusion injury. Free Radic Biol Med. 1989; 7(1):45-52. PubMed PMID: 2753395.
98. Lesnefsky E J, Repine J E, Horwitz L D. Deferoxamine pretreatment reduces canine infarct size and oxidative injury. J Pharmacol Exp Ther. 1990 June; 253(3):1103-9. PubMed PMID: 2359019.

What is claimed is:

1. A method for treating or ameliorating myocardial ischemia or an acute coronary event, comprising administering a therapeutically effective amount of deferiprone or a pharmaceutically acceptable salt thereof to a patient in need thereof.

2. A method for treating or ameliorating intramyocardial hemorrhage or the damage resulting therefrom, comprising administering a therapeutically effective amount of deferiprone or a pharmaceutically acceptable salt thereof to a patient in need thereof, wherein the patient is being treated for myocardial ischemia or an acute coronary event.

3. A method for treating or ameliorating cardiac edema, comprising administering a therapeutically effective amount of deferiprone or a pharmaceutically acceptable salt thereof to a patient in need thereof, wherein the patient is being treated for myocardial ischemia or an acute coronary event.

4. The method of any one of claims 1 to 3, wherein the myocardial ischemia or acute coronary event is an acute myocardial infarction or a ST-segment elevation myocardial infarction (STEMI).

5. The method of any one of claims 1 to 4, wherein the patient is further given a reperfusion therapy.

6. The method of claim 5, wherein the deferiprone or pharmaceutically acceptable salt thereof is administered at a time before, during or after the patient is given the reperfusion therapy.

7. The method of claim 6, wherein the deferiprone or pharmaceutically acceptable salt thereof is administered after the patient is given the reperfusion therapy.

8. A method for treating or ameliorating a myocardial injury, comprising administering a therapeutically effective amount of deferiprone or a pharmaceutically acceptable salt thereof to a patient during or after a reperfusion therapy.

9. The method of claim 8, wherein the patient is at risk for a myocardial injury selected from the group consisting of intramyocardial hemorrhage, cardiac edema, reperfusion arrhythmias, ischemic damage, adverse remodeling of cardiac tissue, and any combination thereof.

10. The method of any one of claims 5 to 9, wherein the reperfusion therapy is a percutaneous coronary intervention (PCI) or a thrombolytic therapy.

11. The method of claim 10, wherein the PCI is coronary angioplasty or insertion of a stent.

12. The method of claim 11, wherein the thrombolytic therapy comprises administering a thrombolytic agent selected from the group consisting of streptokinase, urokinase, alteplase, recombinant tissue plasminogen activator (rtPA), reteplase, tenecteplase, and any combination thereof.

13. The method of any one of claims 1 to 12, wherein the patient further has an ischemia-induced microvascular obstruction.

14. A method of reducing the risk for a myocardial reperfusion injury, comprising administering a therapeutically effective amount of deferiprone or a pharmaceutically acceptable salt thereof to a patient who is at risk of myocardial reperfusion injury.

15. A method for reducing the risk for intramyocardial hemorrhage or the damage resulting therefrom, cardiac edema, or reperfusion arrhythmias, comprising administering a therapeutically effective amount of deferiprone or a pharmaceutically acceptable salt thereof to a patient at risk of intramyocardial hemorrhage, cardiac edema, or reperfusion arrhythmias after suffering a myocardial infarction.

16. The method of claim 14 or 15, wherein the deferiprone or pharmaceutically acceptable salt thereof is administered in combination with a percutaneous coronary intervention (PCI) or a thrombolytic therapy.

17. The method of claim 16, wherein the deferiprone or pharmaceutically acceptable salt thereof is administered before, during or after the percutaneous coronary intervention (PCI) or thrombolytic therapy.

18. The method of claim 16 or 17, wherein the PCI is coronary angioplasty or insertion of a stent.

19. The method of claim 16 or 17, wherein the thrombolytic therapy comprising administering a thrombolytic agent selected from the group consisting of streptokinase, urokinase, alteplase, recombinant tissue plasminogen activator (rtPA), reteplase, tenecteplase, and any combination thereof.

20. The method of any one of claims 1 to 19, wherein said deferiprone or pharmaceutically acceptable salt thereof is administered as part of a pharmaceutical composition comprising a pharmaceutically acceptable carrier.

21. The method of claim 20, wherein said pharmaceutical composition is an immediate release, sustained release or controlled release pharmaceutical composition.

22. The method of any one of claims 1 to 21, wherein said patient has suffered at least one episode of myocardial infarction prior to administration of said deferiprone or pharmaceutically acceptable salt thereof.

23. The method of claim 22, wherein said deferiprone or pharmaceutically acceptable salt thereof is administered less than about twelve hours after the first episode of myocardial infarction.

24. The method of claim 22, wherein said deferiprone or pharmaceutically acceptable salt thereof is administered less than about four hours after the first episode of myocardial infarction.

25. The method of claim 22, wherein said deferiprone or pharmaceutically acceptable salt thereof is administered less than about two hours after the first episode of myocardial infarction.

26. The method of any one of claims 1 to 25, wherein said patient experiences angina, dyspnea on exertion, or congestive heart failure prior to administration of said deferiprone or pharmaceutically acceptable salt thereof.

27. The method of any one of claims 1 to 26, wherein the deferiprone or pharmaceutically acceptable salt thereof is administered to a patient for a first period of time while the patient is suffering from a myocardial infarction and for a second period of time after which the patient has suffered the myocardial infarction.

28. A method of promoting the beneficial remodeling of cardiac tissue in a patient, comprising administering a therapeutically effective amount of deferiprone or a pharmaceutically acceptable salt thereof to a patient before, during or after reperfusion therapy following myocardial ischemia or an acute coronary event in said patient.

29. A method of promoting the beneficial remodeling of cardiac tissue following a surgical or catheter-based revascularization procedure, comprising administering a therapeutically effective amount of deferiprone or a pharmaceutically acceptable salt thereof to a patient undergoing the surgical or catheter-based revascularization procedure.

30. The method of claim 29, wherein the deferiprone or pharmaceutically acceptable salt thereof is administered to the patient for a first period of time prior to and/or during the revascularization procedure and for a second period of time after the patient has completed the revascularization procedure.

31. The method of claim 27, 29 or 30, wherein said deferiprone or pharmaceutically acceptable salt thereof is administered intravenously to said patient during said first period of time.

32. The method of any one of claims 27 and 29 to 31, wherein said deferiprone or pharmaceutically acceptable salt thereof is administered orally to said patient for said second period of time.

33. The method of any one of claims 27 and 29 to 32, wherein said second period of time is at least one week.

34. The method of any one of claims 27 and 29 to 32, wherein said second period of time is one week to six months.

35. The method of any one of claims 28 to 34, wherein said cardiac tissue is injured by surgery.

36. The method of claim 35, wherein said surgery is coronary artery bypass grafting, correction of a congenital heart defect, replacement of a heart valve, or heart transplantation.

37. The method of claim 35 or 36, wherein there is a hemorrhage in said injured cardiac tissue.

38. The method of any one of claims 1 to 37, wherein said deferiprone or pharmaceutically acceptable salt thereof is administered orally or intravenously to said patient.

39. The method of any one of claims 1 to 38, wherein said deferiprone or pharmaceutically acceptable salt thereof is administered in one to six doses per day.

40. The method of any one of claims 1 to 39, wherein said therapeutically effective amount is 1 to 50 mg/kg of deferiprone or equivalent amount of the pharmaceutically acceptable salt thereof administered in one or more oral doses per day up to a maximum of 150 mg/kg/day.

41. The method of any one of claims 1 to 39, wherein said therapeutically effective amount is 1 to 50 mg/kg/day of deferiprone or equivalent amount of the pharmaceutically acceptable salt thereof in an intravenous pharmaceutical composition administered in one or more intravenous doses per day up to a maximum of 150 mg/kg/day.

42. The method of any one of claims 1 to 41, further comprising administering a second chelating agent to said patient.

43. The method of claim 42, wherein said second chelating agent is selected from the group consisting of deferoxamine, deferasirox, desferrithiocin, derivatives thereof, and combinations thereof.

44. The method of any one of claims 1 to 43, further comprising administering an antiplatelet therapy.

45. The method of claim 44, wherein said antiplatelet therapy is selected from the group consisting of aspirin, clopidogrel, prasugrel, ticagrelor, ticopidine, cilostazol, abciximab, eptifibatide, tirofiban, dipyidamole, terutroban, epoprostenol, streptokinase, a plasminogen activator, and combinations thereof.

46. A method of selecting a patient for treatment of a myocardial hemorrhage with deferiprone or a pharmaceutically acceptable salt thereof, comprising determining whether there is a myocardial hemorrhage in the patient after a myocardial infarction.

47. A method of treating or ameliorating a myocardial hemorrhage in a patient, comprising (a) determining whether there is a myocardial hemorrhage in the patient after a myocardial infarction, and (b) administering a therapeutically effective amount of deferiprone, or a pharmaceutically acceptable salt thereof, to said patient if it is determined that there is a hemorrhage at the place of the infarct.

48. The method of claim 15, wherein the patient is at risk for intramyocardial hemorrhage or the damage resulting therefrom.

49. The method of claim 48, wherein said patient exhibits one or more risk indicators for intramyocardial hemorrhage.

50. The method of claim 49, wherein said one or more risk indicators comprise (i) a diagnosis of ST-segment elevation myocardial infarction (STEMI), (ii) an increase in a marker for myocardial damage, (iii) in vivo imaging evidence of an intramyocardial hemorrhage; (iv) a pre-PCI TIMI flow value of 0 or 1, (v) any combination thereof.

51. The method of claim 46 or 47, wherein the determining is carried out by in vivo imaging.

52. The method of claim 50 or 51, wherein said in vivo imaging is by magnetic resonance imaging.

53. The method of claim 50, wherein the marker for myocardial damage is a troponin or creatine kinase.

54. The method of claim 50, wherein the diagnosis of STEMI is determined by an electrocardiogram (ECG).

55. The method of any one of claims 48 to 54, wherein the patient is further given a reperfusion therapy.

56. The method of claim 55, wherein the reperfusion therapy is a percutaneous coronary intervention (PCI) or a thrombolytic therapy.

57. The method of any one of claims 1 to 56, wherein the patient is a human.
